(19) United States
(12) Patent Application Publication
(10) Pub. No.: US 2026/0335683 A1
(43) Pub. Date: Oct. 29, 2026

(54) HOUSING DEVICE, RADIOTHERAPY DEVICE, AND METHOD FOR MOVEMENT CONTROL OF NOZZLE

(71) Applicant: MEVION MEDICAL EQUIPMENT CO., LTD., Suzhou (CN)

(72) Inventors: Junjie ZENG, Suzhou (CN); Ling WANG, Suzhou (CN); Zhihong ZHENG, Suzhou (CN)

(73) Assignee: MEVION MEDICAL EQUIPMENT CO., LTD., Suzhou (CN)

(21) Appl. No.: 19/701,712

(22) Filed: Jun. 8, 2026

Related U.S. Application Data
(63) Continuation-in-part of application No. PCT/CN2024/132929, filed on Nov. 19, 2024.

(30) Foreign Application Priority Data
Dec. 20, 2023 (CN) 202311761422.6

(51) Int. Cl. A61N 5/10

(52) U.S. Cl. CPC A61N 5/1081; A61N 2005/1092

(57) ABSTRACT

A housing device, a radiotherapy device, and a method for movement control of a nozzle are provided. The housing device includes: a support frame assembly and a housing assembly. The support frame assembly includes a fixed base, a movable base plate, and a movable support. The movable base plate is disposed on the fixed base and capable of moving along a first direction, and the movable support is disposed on the movable base plate and capable of moving along the first direction. The housing assembly covers at least a part of the support frame assembly. The housing assembly is provided with a particle beam outlet and includes three or more housings. At least a part of at least one of the housings is capable of entering or leaving at least one other of the housings along the first direction as the movable base plate and/or the movable support moves.

### CROSS-REFERENCE TO RELATED APPLICATIONS

This application is a continuation-in-part of International Patent Application No. PCT/CN2024/132929, filed on Nov. 19, 2024, which claims priority to Chinese Application No. 202311761422.6, filed on Dec. 20, 2023, the entire contents of each of which are incorporated herein by reference.

### TECHNICAL FIELD

The present disclosure relates to the field of high-end medical device technology, for example, to a housing device, a radiotherapy device, and a method for movement control of a nozzle.

### BACKGROUND

Tumors are fatal diseases for human beings and cause great harm to human life and health. Currently, main treatment methods include surgery, radiotherapy, chemotherapy, or the like. With the development of radiobiology, oncology, and medical computers, radiotherapy (radiation therapy) has become increasingly important in tumor treatment.

The housing device of a nozzle of the radiotherapy device is of a fixed type, a single-stage telescopic type, or a single-stage movement mechanism. Due to the limitation of the movement range, in order to accommodate devices such as a dose detection device in the nozzle, the size of the housing device needs to be large, which leads to an excessively large overall volume of the nozzle, consequently occupying too much space in the treatment area, thereby increasing a risk of collision with the nozzle for an operator or a patient. Simultaneously, the excessively large nozzle may cause a sense of oppression to the patient during treatment. Thus, such housing devices need further improvement.

### SUMMARY

An object of the present disclosure is to provide a housing device, a radiotherapy device, and a method for movement control of a nozzle. When the housing device is extended, the radiotherapy device can reach a farther position, increasing a treatment range of the radiotherapy device. When the housing device is retracted, a volume of the radiotherapy device is reduced, which can increase a space of a treatment area, reduce a risk of collision between an operator or a patient and the radiotherapy device, also reduce a sense of oppression of the radiotherapy device on the patient, and improve an experience effect of the patient.

An objective of the present disclosure is achieved by the following technical solutions. One or more embodiments of the present disclosure provide a housing device for installation on an outer frame (also referred to as a drive frame) of a radiotherapy device and capable of passing through a C-shaped inner frame (also referred to as a guide frame) along a front-back direction, comprising: a support frame assembly including a fixed base, a movable base plate, and a movable support, the movable base plate being configured to install an ionization chamber and disposed on the fixed base and capable of moving along an extension direction (also referred to as a first direction) of the support frame assembly, the movable support being disposed on the movable base plate and capable of moving along the extension direction of the support frame assembly, the movable support being configured to install a device for adjusting a particle beam (also referred to as a particle beam adjustment device); a housing assembly covering at least a part of the support frame assembly, the housing assembly being provided with a radiation beam outlet (also referred to as a particle beam outlet) and including three or more housings, wherein two of the housings are capable of at least partially entering or leaving another housing along the extension direction of the support frame assembly as the movable base plate and/or the movable support moves; the housing device is configured to rotate together with the outer frame without moving together with the inner frame.

In some embodiments, the housing device further comprises a drive assembly for driving the movable base plate and the movable support to move along the extension direction of the support frame assembly.

In some embodiments, the drive assembly includes a first gear and a second gear disposed on the movable base plate, a first rack disposed on the fixed base, and a second rack disposed on the movable support, the first rack and the second rack are engaged with the first gear and the second gear respectively, the first gear and the first rack are configured to drive the movable base plate to move along the extension direction of the support frame assembly, and the second gear and the second rack are configured to drive the movable support to move along the extension direction of the support frame assembly.

In some embodiments, a diameter of the second gear is larger than a diameter of the first gear, so that a moving speed of the movable support is greater than a moving speed of the movable base plate.

In some embodiments, the housing device further includes a fixing assembly, the support frame assembly is fixedly connected to the outer frame via the fixing assembly, the support frame assembly is provided with a support frame, the housing assembly includes a first housing, a second housing, and a third housing sequentially arranged along the extension direction of the support frame assembly, and the support frame is connected to the fixed base and the first housing.

In some embodiments, the support frame assembly further includes a first slide rail and a first slide block that are slidably connected, and a second slide rail and a second slide block that are slidably connected, the first slide rail and the first slide block are configured for slidably connecting the movable base plate and the fixed base, and the second slide rail and the second slide block are configured for slidably connecting the movable base plate and the movable support.

In some embodiments, the housing assembly includes the first housing, the second housing, and the third housing sequentially arranged along the extension direction of the support frame assembly, the third housing is located at a first end of the housing device facing a patient, the first housing is fixedly connected to the fixed base, the second housing is slidably connected to the support frame assembly, the third housing is fixedly connected to the movable support, when the housing device is retracted, the second housing is configured to be able to smoothly retract into the first housing, and the third housing is configured to be able to smoothly retract into the second housing.

In some embodiments, the drive assembly further includes a third gear and a third rack meshing with the third gear and disposed on the second housing, and the third gear and the third rack are configured for driving the movable support to move along the extension direction of the support frame assembly.

In some embodiments, a diameter of the third gear is smaller than the diameter of the second gear, so that the moving speed of the movable support is greater than a moving speed of the second housing.

In some embodiments, a pulley is disposed on the movable support, a guide rail slidably connected to the pulley is disposed on the second housing, and the pulley and the guide rail are configured to cause the second housing to perform a linear movement along the extension direction of the support frame assembly.

In some embodiments, a limiting portion is disposed on a second end (also referred to as a connection end) of the fixed base near the outer frame, the limiting portion is configured to limit a distance that the movable base plate moves toward the outer frame side; when the housing device is in a fully retracted state, the movable support is located on a first side of the movable base plate, and the movable base plate abuts against the limiting portion; and when the housing device is in a fully extended state, the movable support is located on a second side of the movable base plate.

One or more embodiments of the present disclosure provide a nozzle, comprising the housing device according to any one of the above, a scanning magnet, an ionization chamber, and a range shifter, where the nozzle is installed on the outer frame and configured to rotate together with the outer frame, the nozzle is provided with cables, and all cables are routed through the outer frame.

One or more embodiments of the present disclosure provides a radiotherapy device, including the nozzle as described above, the outer frame, and the C-shaped inner frame. The outer frame is a rotating frame, including a first mechanical arm and a second mechanical arm located on two sides, and an intermediate arm connecting the first mechanical arm and the second mechanical arm. The intermediate arm is configured for driving the housing device to perform a synchronous rotational movement with the outer frame, and a particle accelerator for providing a particle beam is installed on the intermediate arm.

One or more embodiments of the present disclosure provide a method for moving a nozzle, the nozzle being the nozzle as described above. The method includes: setting an initial state of the nozzle at an initial position to be retracted to a final state along a front-back direction; rotating the outer frame and driving the nozzle to rotate from the initial position to a first position according to a predetermined position to be reached by the nozzle, then extending the nozzle forward from the first position to the predetermined position; after completing particle beam radiation at the predetermined position, retracting the nozzle to the first position.

In some embodiments, the method further includes: after retracting the nozzle to the first position, rotating the outer frame to return the nozzle to the initial position.

Some embodiments of the present disclosure provide a housing device. The housing device is movably disposed on a guide frame of a radiotherapy device and driven by a drive frame of the radiotherapy device. The housing device includes a support frame assembly and a housing assembly. The support frame assembly includes a fixed base, a movable base plate, and a movable support. The movable base plate is disposed on the fixed base and capable of moving along a first direction. The movable support is disposed on the movable base plate and capable of moving along the first direction. The housing assembly covers at least a part of the support frame assembly. The housing assembly is provided with a particle beam outlet and includes three or more housings. At least a part of at least one of the three or more housings is capable of entering or leaving at least one other of the three or more housings along the first direction as the movable base plate and/or the movable support moves.

Some embodiments of the present disclosure provide a radiotherapy device. The radiotherapy device includes a nozzle and a drive frame. The nozzle includes a housing device, an ionization chamber, and a particle beam adjustment device. The housing device is driven by the drive frame. The housing device includes a support frame assembly and a housing assembly. The support frame assembly includes a fixed base, a movable base plate and a movable support. The movable base plate is disposed on the fixed base and capable of moving back and forth along a first direction. The ionization chamber is installed on the movable base plate. The movable support is disposed on the movable base plate and capable of moving back and forth along the first direction. The particle beam adjustment device is installed on the movable support. The housing assembly covers at least a part of the support frame assembly. The housing assembly is provided with a particle beam outlet and includes three or more housings. At least a part of at least one of the three or more housings is capable of entering or leaving at least one other of the three or more housings along the first direction as the movable base plate and/or the movable support moves.

Some embodiments of the present disclosure provide a method for movement control of a nozzle, the method including: setting an initial state of the nozzle at an initial position as being retracted along a first direction to a fully retracted state; driving the nozzle to rotate from the initial position to a first position via a drive frame; controlling the nozzle to extend along the first direction to reach a preset extended state; after the nozzle completes particle beam irradiation at the first position in the preset extended state, controlling the nozzle to retract to the fully retracted state; and driving the nozzle to return from the first position to the initial position via the drive frame.

The embodiments of the present disclosure include but are not limited to the following beneficial effects: the housing device can perform linear movement toward and away from a center of a radiotherapy device by driving the housing assembly to telescopically move together with the support frame assembly, thereby increasing or decreasing a volume of the radiotherapy device. When the housing device is extended, the radiotherapy device can reach a farther position, increasing a treatment range of the radiotherapy device. When the housing device is retracted, the volume of the radiotherapy device is reduced, better achieving miniaturization and integration, increasing a space of a treatment area, reducing a risk of collision between an operator or a patient and the radiotherapy device, also reducing a sense of oppression of the radiotherapy device on the patient, improving an experience effect of the patient, and providing sufficient working space around a top of a treatment couch.

### BRIEF DESCRIPTION OF THE DRAWINGS

The present disclosure will be further described by way of exemplary embodiments, which will be described in detail with reference to the accompanying drawings. These embodiments are not limiting, and in these embodiments, the same reference numerals denote the same structures, wherein:

FIG. 1 is a schematic diagram illustrating a perspective structure of an exemplary housing device according to some embodiments of the present disclosure;

FIG. 2 is a schematic diagram illustrating a structure of a housing device in a fully extended state according to some embodiments of the present disclosure;

FIG. 3 is a schematic diagram illustrating a structure of a housing device in a fully retracted state according to some embodiments of the present disclosure;

FIG. 4 is a schematic diagram illustrating a structure from one perspective when a housing assembly is not installed according to some embodiments of the present disclosure;

FIG. 5 is a partial enlarged view of portion A in FIG. 4;

FIG. 6 is a schematic diagram illustrating a structure from another perspective when the housing assembly is not installed according to some embodiments of the present disclosure;

FIG. 7 is a schematic diagram illustrating a structure of a guide frame and a drive frame from one perspective according to some embodiments of the present disclosure;

FIG. 8 is a schematic diagram illustrating a three-dimensional structure of the drive frame according to some embodiments of the present disclosure

FIG. 9 is a schematic diagram illustrating a structure of the drive frame and the guide frame from another perspective according to some embodiments of the present disclosure;

FIG. 10 is a schematic diagram illustrating a housing device rotating around a central axis of an intermediate arm according to some embodiments of the present disclosure;

FIG. 11 is a schematic diagram illustrating a housing device rotating around a rotation axis according to some embodiments of the present disclosure;

FIG. 12 is a schematic diagram illustrating positions of a gear set and an annular rack according to some embodiments of the present disclosure; and

FIG. 13 is a schematic diagram illustrating an exemplary method for movement control of a nozzle according to some embodiments of the present disclosure.

### LIST OF REFERENCE NUMERALS

100, housing device; 1, support frame assembly; 11, fixed base; 111, first slide block; 112, oblong hole; 113, circular hole; 114, connection end; 12, movable base plate; 121, first slide rail; 122, second slide rail; 123, first side; 124, second side; 13, movable support; 131, second slide block; 132, pulley; 133, guide rail; 14, support frame; 15, limiting portion; 16, treatment end; 20, radiotherapy device; 2, housing assembly; 21, first housing; 22, second housing; 23, third housing; 231, particle beam outlet; 232, grating leaf; 3, drive assembly; 31, first gear; 32, first rack; 33, second gear; 34, second rack; 35, third gear; 36, third rack; 37, fourth gear; 38, fifth gear; 4, fixing assembly; 41, screw; 42, pin; 43, annular fixing assembly; 401, third slide rail; 402, third slide block; 403, annular rack; 404, gear set; 5, frame connector; 200, drive frame; 201, first mechanical arm; 202, second mechanical arm; 203, intermediate arm; 300, guide frame.

### DETAILED DESCRIPTION

Exemplary embodiments will now be described more fully with reference to the accompanying drawings. However, the exemplary embodiments may be embodied in a plurality of forms and should not be construed as limited to the embodiments set forth herein; rather, the embodiments are provided thereby making the present disclosure more thorough and complete, and fully conveying the concept of the exemplary embodiments to those skilled in the art. In the drawings, like reference numerals indicate the same or similar structures, thereby omitting redundant descriptions of the structures.

The terms for expressing positions and directions described in the present disclosure are merely for illustrative purposes with reference to the accompanying drawings. However, modifications may be made as needed, and the changes made are all included within the scope of protection of the present disclosure.

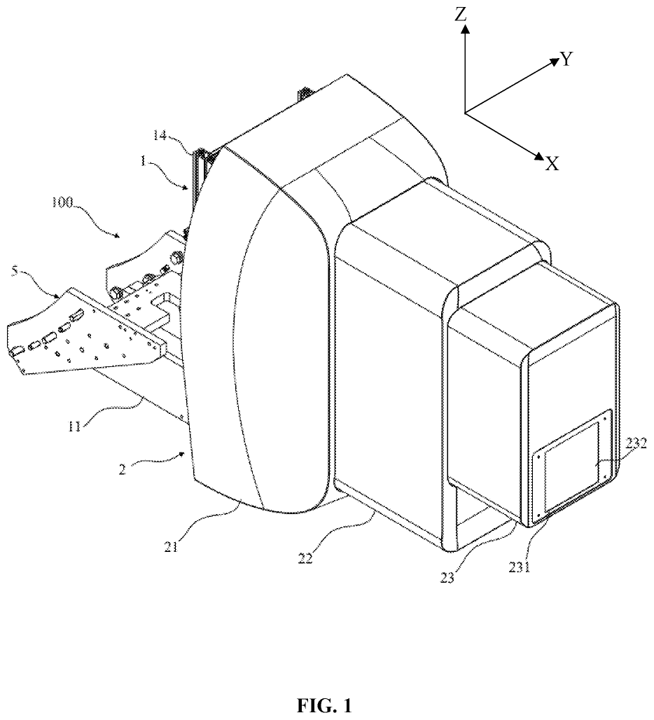

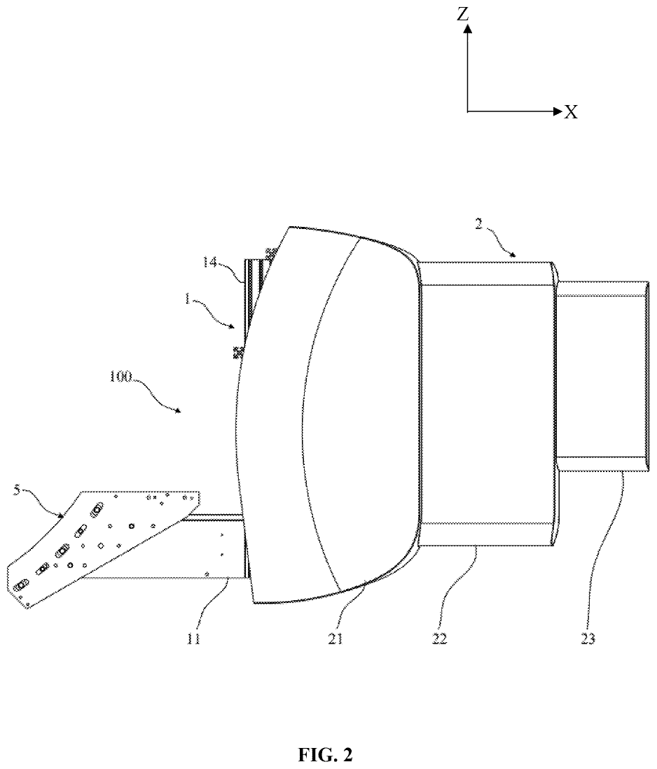

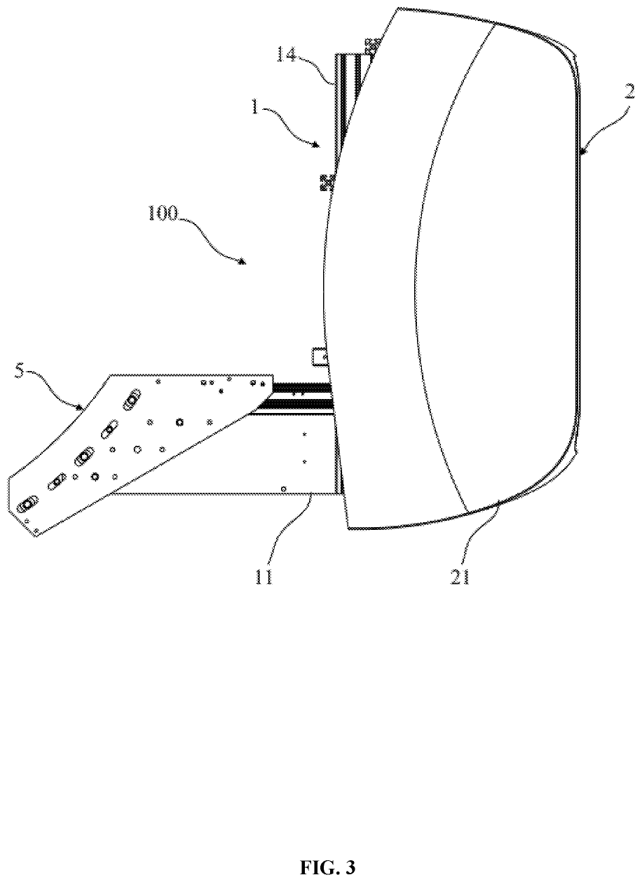

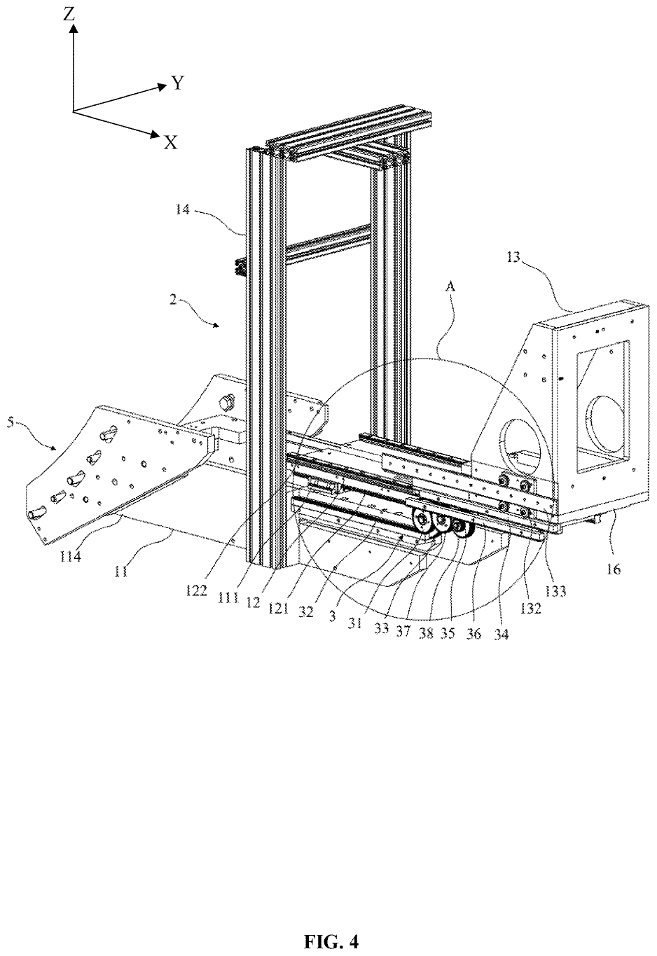

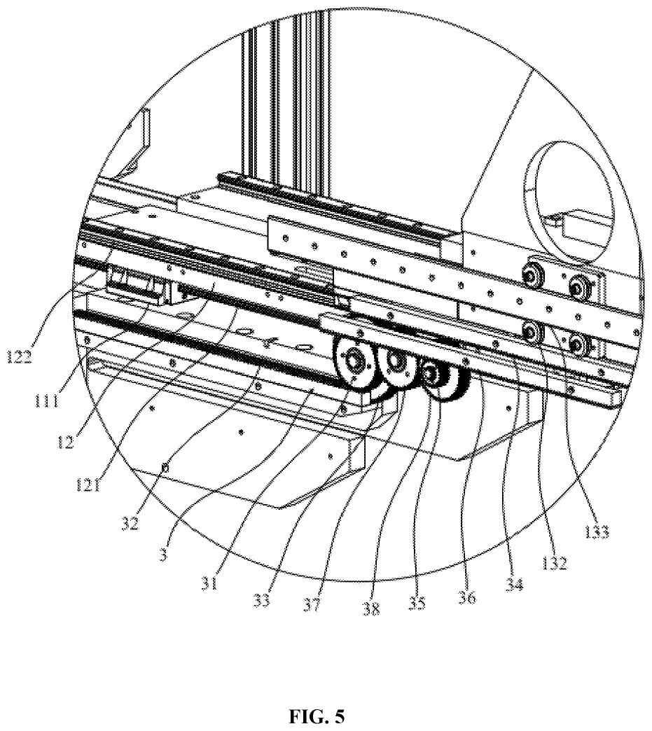

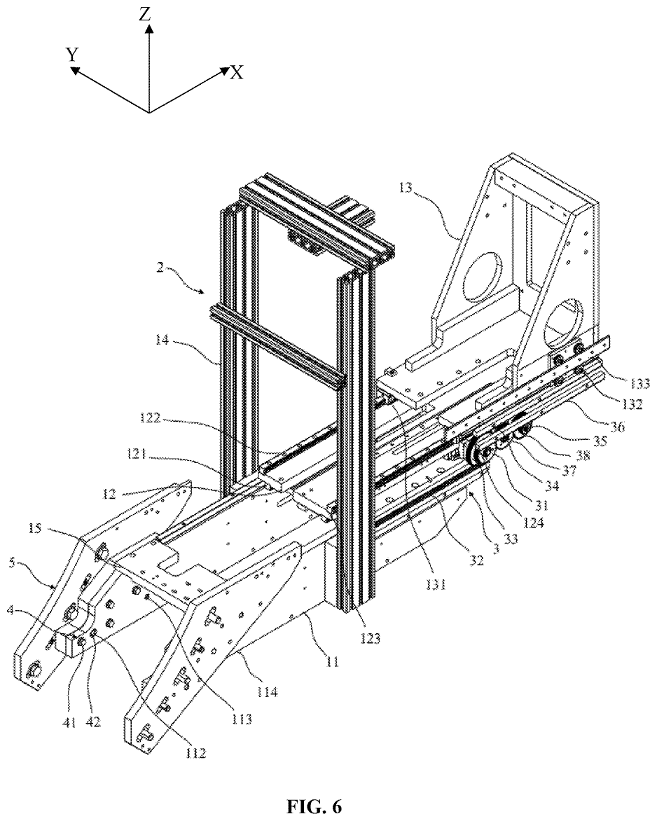

FIG. 1 is a schematic diagram illustrating a perspective structure of an exemplary housing device according to some embodiments of the present disclosure. FIG. 2 is a schematic diagram illustrating a structure of a housing device in a fully extended state according to some embodiments of the present disclosure. FIG. 3 is a schematic diagram illustrating a structure of a housing device in a fully retracted state according to some embodiments of the present disclosure. FIG. 4 is a schematic diagram illustrating a structure from one perspective when a housing assembly is not installed according to some embodiments of the present disclosure. FIG. 5 is a partial enlarged view of portion A in FIG. 4. FIG. 6 is a schematic diagram illustrating a structure from another perspective when the housing assembly is not installed according to some embodiments of the present disclosure.

In some embodiments, as shown in FIGS. 1-6, a housing device 100 includes a support frame assembly 1 and a housing assembly 2. The support frame assembly 1 includes a fixed base 11, a movable base plate 12, and a movable support 13. The movable base plate 12 is disposed on the fixed base 11 and capable of moving back and forth along a first direction, and the movable support 13 is disposed on the movable base plate 12 and capable of moving back and forth along the first direction. The housing assembly 2 covers at least a part of the support frame assembly 1. The housing assembly 2 is provided with a particle beam outlet 231 and includes three or more housings. At least a part of at least one of the three or more housings is capable of entering or leaving at least one other of the three or more housings along the first direction as the movable base plate 12 and/or the movable support 13 moves.

The housing device 100 is configured to enclose and protect internal core components of a radiotherapy device 20.

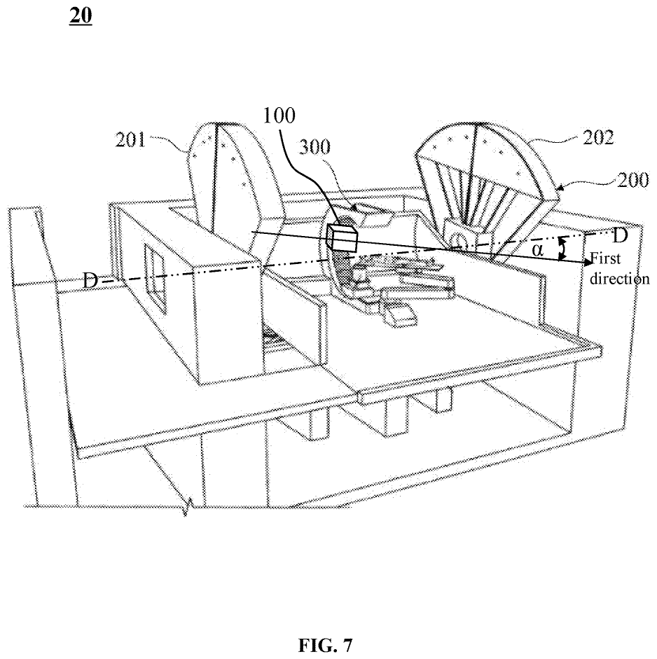

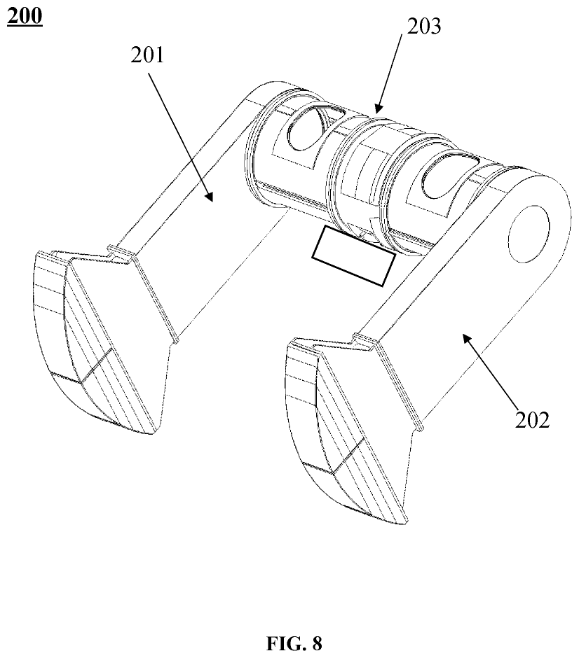

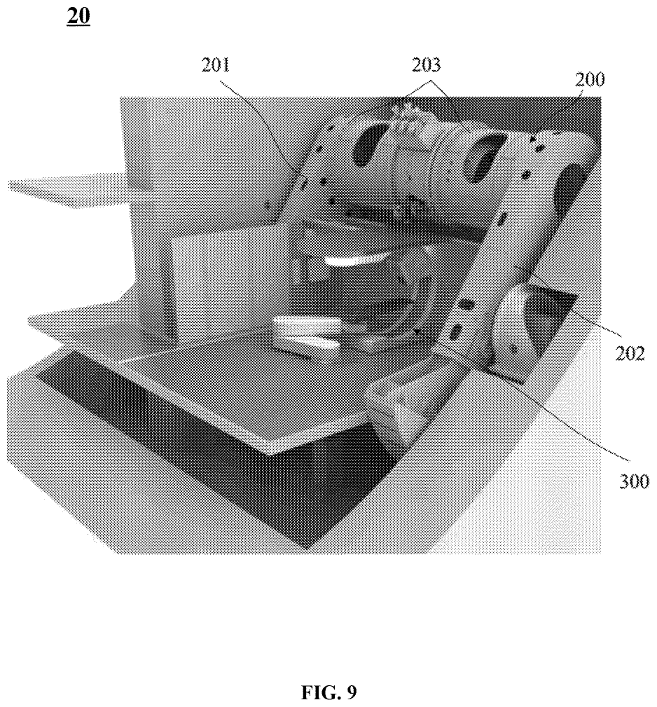

More descriptions regarding the radiotherapy device 20, a guide frame 300, and a drive frame 200 may be found elsewhere in the present disclosure (e.g., FIGS. 7-9 and related descriptions thereof).

In some embodiments, the housing device 100 is movably arranged on the guide frame 300 of the radiotherapy device 20 and driven by the drive frame 200 of the radiotherapy device 20.

In some embodiments, the housing device 100 may be movably arranged on the guide frame 300 in a plurality of manners. For example, the housing device 100 is movably arranged by matching a slide block disposed at the bottom thereof with a guide rail on the guide frame 300; and the drive frame 200 drives the housing device 100 to move along the guide rail via a gear and rack transmission mechanism.

In some embodiments, the housing device 100 may also be movably arranged on the guide frame 300 in other feasible manners, e.g., by matching a roller assembly with a guide slot to achieve the movable arrangement; but the present disclosure is not limited thereto.

In some embodiments, the drive frame 200 may drive the housing device 100 to move on the guide frame 300 in a plurality of manners. For example, the drive frame 200 drives the housing device 100 to move along the guide rail on the guide frame 300 via the gear and rack transmission mechanism. As another example, the drive frame 200 is connected to a lead screw nut mechanism via a servo motor, thereby driving the housing device 100 to move. The driving manner is not limited herein.

The support frame assembly 1 is configured to support the housing assembly 2 and provide movement guidance.

The fixed base 11 is a basic load-bearing component in the support frame assembly 1, configured to provide a fixed foundation for other components of the housing device 100 (e.g., the housing assembly 2).

The movable base plate 12 is configured to connect the fixed base 11 and the movable support 13, and provide a movement guiding function for the movable support 13.

The movable support 13 is configured to achieve a secondary movement or to move with the movement of the movable base plate 12.

The first direction refers to the telescopic direction of the support frame assembly 1. For example, as shown in FIGS. 1-6, the first direction may be a positive direction of the X-axis or a negative direction of the X-axis. The following descriptions take as an example that the first direction is the positive direction of the X-axis, the support frame assembly 1 extends along the positive direction of the X-axis, and retracts along the negative direction of the X-axis.

In some embodiments, the fixed base 11, the movable base plate 12, and the movable support 13 may be connected in a plurality of manners. For example, high-precision linear guide pairs are configured between the movable base plate 12 and the fixed base 11, and between the movable support 13 and the movable base plate 12. The movable base plate 12 slides along a linear guide rail on the fixed base 11, and the movable support 13 then slides along a linear guide rail on the movable base plate 12, thereby achieving a smooth movement in two stages along the first direction. As another example, a physical structure in which dovetail grooves and dovetail blocks are engaged in a sliding fit is adopted between the movable base plate 12 and the fixed base 11, and between the movable support 13 and the movable base plate 12, and a self-lubricating material is coated on contact surfaces to achieve movement along the first direction.

In some embodiments, the fixed base 11, the movable base plate 12, and the movable support 13 may also be connected in other feasible manners, and the present disclosure is not limited thereto.

The support frame assembly 1 is configured to implement a multi-layer nesting telescopic movement support structure by sliding the movable base plate 12 relative to the fixed base 11 and sliding the movable support 13 relative to the movable base plate 12.

The housing assembly 2 refers to the protective housing of housing components that are spliced or nested from the plurality of housings. A housing is a single independent shielding unit that forms the housing assembly 2.

In some embodiments, the housing assembly 2 includes three or more housings. In some embodiments, the housing assembly 2 includes four or more housings. In some embodiments, the housing assembly 2 includes five or more housings.

In some embodiments, the housing assembly 2 covers at least a part of the support frame assembly 1. In some embodiments, the housing assembly 2 completely covers the support frame assembly 1.

The particle beam outlet 231 is configured for an adjusted particle beam to be emitted to achieve radiotherapy. In some embodiments, the particle beam outlet 231 is a hole penetrating the housing assembly 2. In some embodiments, the particle beam outlet 231 may be a region composed of a transparent material for rays to be emitted. The transparent material may be plastic, glass, or the like.

In some embodiments, the housing assembly 2 includes three housings. In some embodiments, the housing assembly 2 includes four housings. In some embodiments, the housing assembly 2 includes five or more housings.

In some embodiments, the plurality of housings are coaxially nested with each other, and when the movable base plate 12 or the movable support 13 moves along the first direction, at least a part of at least one of the housings may enter or leave at least one other of the housings in a coaxially nested manner.

In some embodiments, the plurality of housings are hinged to each other, presenting a layered housing. When the movable base plate 12 or the movable support 13 moves along the first direction, the housings, by matching side slide slots and guide pin shafts, slide and overlap with each other in parallel along the first direction, or gradually unfold and separate.

In some embodiments, the housing device 100 shown in FIGS. 1-6 of the present disclosure may be installed on the drive frame 200 of the radiotherapy device and may pass through the guide frame 300 in the front-back direction. In some embodiments, the housing device 100 includes the support frame assembly 1 and the housing assembly 2.

In some embodiments, with reference to FIGS. 4-6, the support frame assembly 1 includes the fixed base 11, the movable base plate 12, and the movable support 13. The movable base plate 12 is configured to install an ionization chamber (not shown), is disposed on the fixed base 11, and is capable of moving back and forth along the first direction. The movable support 13 is disposed on the movable base plate 12 and is capable of moving back and forth along the first direction (i.e., the front-back direction). The movable support 13 is configured to install a particle beam adjustment device. The particle beam adjustment device includes devices such as an adaptive aperture (AA) for the proton beam or a range shifter (RS). A rear side of the movable support 13 (i.e., the side away from a patient) is also provided with the ionization chamber (not shown).

The support frame assembly 1 adopts a double-layer movement structure, thereby allowing the support frame assembly 1 to have a larger retraction stroke. Specifically, when the support frame assembly 1 extends along a length direction of the fixed base 11, it may reach a farther position, and when the support frame assembly 1 retracts, it can reduce an overall size of the support frame assembly 1. Compared to a single-layer movement structure, when the support frame assembly 1 of the present disclosure is fully retracted, the support frame assembly 1 has a smaller volume and can release more space. When the support frame assembly 1 is fully extended, the support frame assembly 1 may reach a farther position, thereby achieving a larger treatment range. The aforementioned extension/retraction direction refers to the front-back direction (i.e., the first direction). For example, when the movable support 13 extends, it moves forward towards the patient, and when the movable support 13 retracts, it moves backward away from the patient. Specifically, the side close to the patient (or away from the frame connector 5) is a front side, and the side away from the patient (or close to the frame connector 5) is a rear side.

In some embodiments, the movable base plate 12 is disposed above the fixed base 11, and the movable support 13 is disposed above the movable base plate 12. When the support frame assembly 1 is fully extended, a part of the movable base plate 12 extends to the right side and may protrude from the fixed base 11, i.e., a part of the movable base plate 12 extends toward the side close to the patient and may protrude from the fixed base 11. The movable support 13 is on the right side of the movable base plate 12 and may protrude from the movable base plate 12, i.e., the movable support 13 is disposed on the side of the movable base plate 12 close to the patient and may protrude from the movable base plate 12. When the support frame assembly 1 is fully retracted, the movable base plate 12 retracts to the left side, i.e., retracts toward the side away from the patient. The right side of the movable base plate 12 is substantially flush with or retracted into the right side of the fixed base 11. The movable support 13 is on the left side of the movable base plate 12, i.e., on the side of the movable base plate 12 away from the patient. Furthermore, the right side of a treatment device installed on the movable support 13 is substantially flush with or retracted into the right side of the fixed base 11.

The “upper”, “lower”, “left”, “right”, “front”, and “rear” sides described in the present disclosure are relative concepts. As shown in FIGS. 1-6, a positive direction of the Z-axis is defined as “upper”, and a negative direction of the Z-axis is defined as “lower”. The positive direction of the X-axis is defined as “right” or “front”, i.e., the direction close to the patient. The negative direction of the X-axis is defined as “left” or “rear”, i.e., the direction away from the patient.

In some embodiments, a grating leaf 232 may be disposed at the rear side of the particle beam outlet 231. The grating leaf 232 is composed of a plurality of narrow and long metal plates. The metal plates may be moved at small intervals and in precise positions to control the shape and intensity of a focused particle beam (also referred to as a radiation beam), to achieve a treatment effect. The conventional grating leaf 232 typically requires manual adjustment, while the adaptive grating leaf 232 can automatically monitor and adjust a dose distribution and a position during a radiotherapy process. In the present embodiment, the grating leaf 232 is an adaptive grating leaf. The adaptive grating leaf, through integration with a radiotherapy planning system and an image-guided system, can monitor changes in tumors and surrounding tissues in real-time and adjust the adaptive grating leaf. The adaptive grating leaf contributes to reducing errors and improving treatment accuracy, and may also reduce the number of radiotherapy sessions and doses the patient needs to receive, thereby reducing the risk of side effects and complications brought to the patient by radiotherapy.

In some embodiments, the housing device 100 includes a fully extended state and a fully retracted state. The fully extended state refers to a state where the housing device 100 is fully expanded and has a maximum size along the first direction. The fully retracted state refers to a state where the housing device 100 is fully retracted and has a minimum size along the first direction. In the fully extended state, the movable base plate 12 moves along the first direction (e.g., the positive direction of the X-axis) and away from the fixed base 11, the movable support 13 moves along the first direction and away from the movable base plate 12, and the plurality of housings move away from each other (as shown in FIGS. 1-2). In the fully retracted state, the movable base plate 12 moves along a direction opposite to the first direction (e.g., the negative direction of the X-axis) and approaches the fixed base 11, the movable support 13 moves along the direction opposite to the first direction and approaches the movable base plate 12, and the plurality of housings enter each other (as shown in FIG. 3).

According to some embodiments of the present disclosure, by providing the support frame assembly 1, which includes the fixed base 11, the movable base plate 12, and the movable support 13, and has a double-layer sliding structure, in cooperation with the housing assembly 2, which includes three or more housings capable of telescopically nesting with each other, a movement flexibility of the radiotherapy device is improved. When extending, the housing device 100 can significantly increase the treatment range. When retracting, it effectively reduces an overall volume, thereby releasing more space in a treatment area and reducing a risk of collision between the operator or the patient and the device. Meanwhile, it alleviates a sense of compression experienced by the patient during treatment, improves an experience effect, and provides sufficient working space around a top of a treatment bed.

In conventional devices, the housing device 100 is arranged on a C-shaped rail of the guide frame 300 and driven by a motor. The housing device 100 and the drive frame 200 move independently. Therefore, there is always a time difference between the movement of the housing device 100 and the movement of the drive frame 200. Furthermore, the guide frame 300 requires a separate cable system. Compared to the housing device 100 being arranged on the C-shaped rail of the guide frame 300, directly fixing the housing device 100 to the drive frame 200 allows the housing device 100 to rotate synchronously with the rotation of the drive frame 200, thereby avoiding the time difference between the movement of the housing device 100 and the movement of the drive frame 200. This arrangement also makes the structure of the housing device 100 very simple, allowing the cable drag chain originally on the guide frame 300 to be eliminated. All cables on the housing device 100 are routed through the drive frame 200, which integrates and optimizes the wiring structure of the entire radiotherapy device, thereby significantly reducing debugging costs and manufacturing costs. This arrangement eliminates the C-shaped guide rail on the guide frame 300, thereby making the structure of the guide frame 300 very simple and reducing many debugging costs. Furthermore, the cable drag chain originally on the guide frame 300 is eliminated, and the cables on a nozzle (also referred to as a treatment head) are configured to be routed through the drive frame 200, which integrates and optimizes the wiring structure of the entire radiotherapy device, thereby significantly reducing debugging costs and manufacturing costs.

In some embodiments, as shown in FIG. 6, the housing device 100 further includes a fixing assembly 4. The support frame assembly 1 is fixedly connected to the drive frame 200 via the fixing assembly 4.

The fixing assembly 4 is configured to fixedly connect the support frame assembly 1 to the drive frame 200.

In some embodiments, the fixing assembly 4 may include a bolt and a nut. Mounting holes are formed on the support frame assembly 1 and the drive frame 200. The bolt passes through the mounting holes of the support frame assembly 1 and the drive frame 200, and is tightened by the nut, thereby achieving a fixed connection therebetween.

In some embodiments, the fixing assembly 4 may further include a snap-fit structure. An elastic detent pin is provided on the support frame assembly 1, and a matching slot is provided on the drive frame 200. The elastic detent pin snaps into the slot to achieve a quick fixed connection.

In some embodiments, the fixing assembly 4 may also be other feasible structures, such as a clamp-type connector, a flange, or the like, but is not limited thereto.

In some embodiments, as shown in FIG. 6, the support frame assembly 1 of the housing device 100 is fixedly connected to a frame connector (also referred to as an outer frame connector) 5 via the fixing assembly 4. The frame connector 5 refers to a component for connecting the housing device 100 to the drive frame 200. In some embodiments, the fixing assembly 4 may include a screw 41 and a pin 42. The pin 42 is configured to provide positioning between the fixing assembly 4 and the frame connector 5, to facilitate installation and removal between the fixing assembly 4 and the frame connector 5. The screw 41 is configured to fixedly connect the fixing assembly 4 and the frame connector 5. The fixed base 11 and the frame connector 5 may further be provided with an oblong hole 112 and a circular hole 113. The oblong hole 112 facilitates fine adjustment of the position between the fixed base 11 and the frame connector 5, thereby eliminating machining errors between parts and improving installation accuracy between the fixed base 11 and the frame connector 5.

In some embodiments, as shown in FIGS. 1-6, the support frame assembly 1 further includes a support frame 14. The housing assembly 2 includes a first housing 21, a second housing 22, and a third housing 23 sequentially arranged along the first direction. The support frame 14 is connected to the fixed base 11 and the first housing 21.

The support frame 14 is configured to carry and support an internal structure of the housing device 100. In some embodiments, the support frame 14 includes at least one of a metal material, a carbon fiber material, or the like. The support frame 14 may support and maintain a shape of the first housing 21.

In some embodiments, the support frame 14 may be L-shaped, triangular, or a frame structure. An end of the support frame 14 is fixedly connected to the fixed base 11, and the other end is fixedly connected to an inner wall of the first housing 21. The fixed connection manner includes at least one of welding, threaded connection (e.g., a screw or a bolt), riveting, integral molding, or the like.

By providing the support frame 14, on one hand, the structural strength of the fixed base 11 may be improved, and on the other hand, more stable support may be provided for the first housing 21, ensuring the structural strength of the first housing 21, and further improving the overall structural strength of the housing device 100.

The first housing 21 is configured to provide positioning and a space for retraction. In the fully retracted state, the first housing 21 is located on an outer layer of the housing assembly 2.

The second housing 22 is adjacent to the first housing 21, and is located on a middle layer of the housing assembly 2 in the fully retracted state. In some embodiments, the second housing 22 is configured to accommodate the movable support 13.

The third housing 23 is adjacent to the second housing 22, and is located on an inner layer of the housing assembly 2 in the fully retracted state. In some embodiments, the third housing 23 is located at a treatment end 16 (also referred to as a first end) of the housing device 100 facing the patient, and is configured to accommodate the particle beam adjustment device. The treatment end 16 refers to an end of the housing device 100 close to the patient during treatment.

In some embodiments, the particle beam outlet 231 is provided at an end of the third housing 23 facing the patient. A particle beam (also referred to as a radiation beam) is emitted through the particle beam outlet 231 and reaches an affected area of the patient. The particle beam may be a proton beam.

In some embodiments, the first housing 21, the second housing 22, and the third housing 23 overlap each other via stepped surfaces at edges thereof, and are connected by a sealing strip for splicing and transition.

In some embodiments, the first housing 21, the second housing 22, and the third housing 23 may also be slidably connected via guide rails. Guiding slide rails are provided on side surfaces of the housings. The housings are pushed into the respective slide rails along the first direction and locked by internal limiting portions.

In some embodiments, the sequential arrangement of the first housing 21, the second housing 22, and the third housing 23 may also be achieved in other manners. For example, magnetic adsorption alignment may be performed based on mechanical limiting rules; relevant spacing parameters may be adjusted according to scene configurations; module components may also be centrally deployed or distributedly deployed, but are not limited thereto.

In some embodiments, as shown in FIGS. 1, 2, and 4, two housings (e.g., the second housing 22 and the third housing 23) may at least partially enter or leave another housing (e.g., the first housing 21) along the first direction as the movable base plate 12 and/or the movable support 13 moves. More descriptions regarding the first housing 21, the second housing 22, and the third housing 23 may be found in the following related descriptions.

According to some embodiments of the present disclosure, by providing the fixing assembly 4 to fixedly connect the support frame assembly 1 to the drive frame 200, the housing device 100 is allowed to rotate synchronously with the drive frame 200, thereby avoiding problems of asynchronous movement between the nozzle and the drive frame 200 in conventional structures. Furthermore, by providing the support frame 14 connected to the fixed base 11 and the first housing 21, the structural strength of the fixed base 11 is effectively enhanced, and stable support is provided for the first housing 21, thereby improving overall structural stability and reliability of the housing device 100, and further ensuring operational accuracy and safety of the device during radiotherapy.

In some embodiments, as shown in FIG. 4 to FIG. 6, the support frame assembly 1 further includes a first slide rail 121 and a first slide block 111 slidably connected, and a second slide rail 122 and a second slide block 131 slidably connected. The movable base plate 12 and the fixed base 11 are slidably connected via the first slide rail 121 and the first slide block 111, and the movable base plate 12 and the movable support 13 are slidably connected via the second slide rail 122 and the second slide block 131.

The first slide rail 121 and the first slide block 111 are configured to slidably connect the movable base plate 12 and the fixed base 11, thereby facilitating a smoother movement between the movable base plate 12 and the fixed base 11. The first slide block 111 may relatively slide along the first direction in the first slide rail 121. In some embodiments, the first slide rail 121 may be disposed on the fixed base 11, and the first slide block 111 may be disposed on the movable base plate 12. In other embodiments, the first slide rail 121 may be disposed on the movable base plate 12, and the first slide block 111 may be disposed on the fixed base 11.

The second slide rail 122 and the second slide block 131 are configured to slidably connect the movable base plate 12 and the movable support 13, thereby facilitating the smoother movement between the movable base plate 12 and the movable support 13. The second slide block 131 may relatively slide along the first direction in the second slide rail 122. In some embodiments, the first slide rail 121 is disposed on the side of the movable base plate 12 facing the fixed base 11, and the first slide block 111 is disposed on the side of the fixed base 11 facing the movable base plate 12. In other embodiments, the first slide rail 121 is disposed on the side of the fixed base 11 facing the movable base plate 12, and the first slide block 111 is disposed on the side of the movable base plate 12 facing the fixed base 11.

In some embodiments, a plurality of the first slide rails 121 and a plurality of the first slide blocks 111 may be provided to make the connection between the movable base plate 12 and the fixed base 11 more stable, while ensuring the smoother movement between the movable base plate 12 and the fixed base 11. The first slide rail 121 is disposed on the movable base plate 12, and the first slide block 111 is disposed on the fixed base 11, or the first slide rail 121 is disposed on the fixed base 11, and the first slide block 111 is disposed on the movable base plate 12. In some embodiments, the count of the first slide rails 121 is two, the two first slide rails 121 extend along the first direction, the count of the first slide blocks 111 is four, the two first slide rails 121 are respectively disposed on two sides of a surface of the movable base plate 12 facing the fixed base 11, and the four first slide blocks 111 are respectively disposed on four corners of the fixed base 11.

In some embodiments, a plurality of the second slide rails 122 and a plurality of the second slide blocks 131 may be provided to make the connection between the movable base plate 12 and the movable support 13 more stable, while ensuring the smoother movement between the movable base plate 12 and the movable support 13. The second slide rail 122 is disposed on the movable base plate 12, and the second slide block 131 is disposed on the movable support 13, or the second slide rail 122 is disposed on the movable support 13, and the second slide block 131 is disposed on the movable base plate 12. In some embodiments, the count of the second slide rails 122 is two, the two second slide rails 122 extend along the first direction of the support frame assembly 1, the count of the second slide blocks 131 is four, the two second slide rails 122 are respectively disposed on two sides of a surface of the movable base plate 12 facing the movable support 13, and the four second slide blocks 131 are respectively disposed on four corners of the movable support 13.

According to some embodiments of the present disclosure, by disposing the first slide rail 121 and the first slide block 111 between the movable base plate 12 and the fixed base 11, and disposing the second slide rail 122 and the second slide block 131 between the movable base plate 12 and the movable support 13, stable and precise guiding of the double-layer sliding structure is achieved, thereby significantly improving the smoothness and positioning accuracy of the telescopic movement of the support frame assembly 1, and ensuring the stability and reliability of the ionization chamber and the particle beam adjustment device during movement, thereby guaranteeing the accuracy of radiotherapy, while reducing motion friction and mechanical wear, and extending the service life of the device.

In some embodiments, as shown in FIGS. 4-6, the housing device 100 further includes a drive assembly 3. The movable base plate 12 and the movable support 13 are configured to move back and forth along the first direction (e.g., the positive direction of the X-axis) driven by the drive assembly 3.

The drive assembly 3 refers to a power transmission mechanism configured to drive the movable base plate 12 and the movable support 13 to perform a telescopic movement. In some embodiments, the drive assembly 3 includes a motor (not shown in the figures) and a transmission structure. The motor includes, but is not limited to, a servo motor, a stepping motor, a DC brushless motor, a linear motor, an AC geared motor, or the like. The transmission structure includes, but is not limited to, a gear-rack transmission, a lead screw-nut transmission, a synchronous belt transmission, a chain-sprocket transmission, or the like.

In some embodiments, under the energy supply of the motor of the drive assembly 3, the transmission of the transmission structure drives the movable base plate 12 to move back and forth along the first direction relative to the fixed base 11, and simultaneously drives the movable support 13 to move back and forth along the first direction relative to the movable base plate 12.

In some embodiments, as shown in FIGS. 4-6, the drive assembly 3 is configured to drive the movable base plate 12 and the movable support 13 to move back and forth along the first direction, that is, the drive assembly 3 may drive the support frame assembly 1 to perform a telescopic movement. In some embodiments, the drive assembly 3 includes a motor. The motor may accurately cause the radiotherapy device to reach the predetermined position for radiotherapy, and may also accurately reduce the volume of the radiotherapy device, thereby increasing the space in a treatment area.

According to some embodiments of the present disclosure, by configuring the drive assembly 3 to drive the movable base plate 12 and the movable support 13 to move along the first direction, automated and precise telescopic control of the double-layer structure of the support frame assembly 1 is achieved. Compared with manual adjustment or single-stage driving, the drive assembly 3 is capable of flexibly controlling the extension and retraction stroke and speed according to treatment requirements, thereby ensuring that the ionization chamber and the particle beam adjustment device quickly and stably reach the predetermined position, thereby improving the response efficiency and treatment accuracy of the radiotherapy device.

Meanwhile, adopting the drive assembly 3 to replace manual operation or complex external drive mechanisms simplifies the device operation process, reduces the risk of errors caused by human intervention, and provides a strong guarantee for the safety and reliability of the radiotherapy process.

In some embodiments, as shown in FIGS. 4-6, the drive assembly 3 includes a first gear 31 and a second gear 33, a first rack 32 disposed on the fixed base 11, and a second rack 34 disposed on the movable support 13. The first rack 32 and the second rack 34 are engaged with the first gear 31 and the second gear 33, respectively. An extension direction of the first rack 32 and an extension direction of the second rack 34 are both parallel to the first direction. The movable base plate 12 is driven by the first gear 31 and the first rack 32 to move back and forth along the first direction, and the movable support 13 is driven by the second gear 33 and the second rack 34 to move back and forth along the first direction.

In some embodiments, the first gear 31 and the second gear 33 are coaxially connected to the output shaft of the motor of the drive assembly 3, and respectively cooperate with the first rack 32 and the second rack 34 to transmit mechanical power.

In some embodiments, the first rack 32 is fixedly connected to the fixed base 11, and the second rack 34 is fixedly connected to the movable support 13. A manner of fixed connection may be welding, bonding, or snap connection, or the like, which is not limited thereto.

In some embodiments, as shown in FIGS. 4-6, the first gear 31 and the first rack 32 are configured to drive the movable base plate 12 to move back and forth along the first direction, and the second gear 33 and the second rack 34 are configured to drive the movable support 13 to move back and forth along the first direction. In some embodiments, the first gear 31 and the second gear 33 are coaxially arranged, and may be rotatably installed on the movable base plate 12 via a bearing. The first gear 31 and the second gear 33 are connected to the motor, and a single motor may drive the first gear 31 and the second gear 33 to rotate simultaneously, thereby reducing the count of motors and the space occupied by the motor, which in turn reduces the volume of the housing assembly 2, which further reduces the volume of the radiotherapy device, and lowers the manufacturing cost of the radiotherapy device.

According to some embodiments of the present disclosure, by adopting a double-layer independent but coaxially linked gear-rack transmission structure in which the first gear 31 is engaged with the first rack 32 to drive the movable base plate 12, and the second gear 33 is engaged with the second rack 34 to drive the movable support 13, a coordinated telescopic movement of the movable base plate 12 and the movable support 13 along the first direction is achieved. This structure allows both to independently control their respective strokes and speeds, and also maintain motion coordination under the drive of a single power source, thereby avoiding synchronization control difficulties caused by a plurality of drive sources. Meanwhile, the gear-rack transmission manner has advantages such as high transmission efficiency, good positioning accuracy, and strong load-bearing capacity, and is capable of ensuring that the ionization chamber and the particle beam adjustment device stably and accurately reach the predetermined position during extension and retraction, thereby effectively improving the treatment accuracy and operational reliability of the radiotherapy device and simplifying the structural design and control logic of the drive system.

In some embodiments, as shown in FIG. 5, the diameter of the second gear 33 is larger than the diameter of the first gear 31.

In some embodiments, by configuring the diameter of the second gear 33 to be larger than the diameter of the first gear 31, the movement speed of the movable support 13 may be greater than the movement speed of the movable base plate 12. When the first gear 31 and the second gear 33 are coaxially arranged, that is, the rotational angular velocities of the first gear 31 and the second gear 33 are the same, the distance that the movable support 13 needs to move while driving the third housing 23 is longer than the distance that the movable base plate 12 needs to move, thus, the diameter of the second gear 33 is larger than the diameter of the first gear 31. The diameter of the second gear 33 and the diameter of the first gear 31 may be set according to actual needs, to allow the movable support 13 and the movable base plate 12 to simultaneously reach their respective predetermined positions when the housing device 100 retracts.

According to some embodiments of the present disclosure, by configuring the diameter of the second gear 33 to be larger than that of the first gear 31, differential driving between the movable support 13 and the movable base plate 12 is achieved, thereby allowing the movable support 13 to obtain the faster movement speed than the movable base plate 12 under a single power source input, thereby matching the different extension and retraction stroke requirements of both, and ensuring that the movable support 13 and the movable base plate 12 can simultaneously reach their predetermined positions during retraction or extension of the housing device 100, avoiding motion interference or stroke errors, and improving the coordination, control accuracy, and operational efficiency of the extension and retraction movement.

In some embodiments, as shown in FIGS. 1-2, the housing assembly 2 includes the first housing 21, the second housing 22, and the third housing 23 sequentially arranged along the first direction, the first housing 21 is fixedly connected to the fixed base 11, the second housing 22 is slidably connected to the support frame assembly 1, and the third housing 23 is fixedly connected to the movable support 13. When the housing device 100 retracts, the second housing 22 retracts into the first housing 21, and the third housing 23 retracts into the second housing 22.

In some embodiments, the fixed connection includes a thread connection, a snap connection, welding, or the like.

In some embodiments, a manner in which the second housing 22 is slidably connected to the movable base plate 12 of the support frame assembly 1 may be a cooperation between a guide rail and a slide block or a cooperation between a linear bearing and a guide rod, but is not limited thereto.

In some embodiments, as shown in FIG. 4, a pulley 132 is disposed on the movable support 13, a guide rail 133 slidably connected with the pulley 132 is disposed on the second housing 22, and the second housing 22 performs a linear movement along the first direction driven by the pulley 132 and the guide rail 133.

The pulley 132 and the guide rail 133 are configured to cooperate to achieve the movement of the second housing 22 along the first direction.

In some embodiments, the count of the pulleys 132 is greater than one, e.g., four, and the four pulleys 132 are arranged in a quadrilateral shape. Two pulleys 132 are respectively disposed on both sides of the guide rail 133, and the four pulleys 132 may constrain the guide rail 133 to perform a linear movement along the first direction, which in turn ensures that the second housing 22 performs the linear movement along the first direction. The pulley 132 and the guide rail 133 also have a supporting effect on the second housing 22, which may improve the stability of the second housing 22 and avoid shaking of the second housing 22 during movement.

In some embodiments, referring to FIGS. 2 and 3, the opening size of the first housing 21 is larger than the opening size of the second housing 22, thereby allowing the second housing 22 to at least partially enter or leave the first housing 21 along the first direction. The opening size of the second housing 22 is larger than the opening size of the third housing 23, thereby allowing the third housing 23 to at least partially enter or leave the second housing 22 along the first direction of the support frame assembly 1. At least a part of the second housing 22 and the third housing 23 can retract into the first housing 21, to reduce the overall volume of the housing assembly 2. In some embodiments, the length of the first housing 21 along the first direction is greater than or equal to the length of the second housing 22 along the first direction, and the length of the second housing 22 along the first direction is greater than or equal to the length of the third housing 23 along the first direction. According to the above arrangement manner, the second housing 22 and the third housing 23 can fully retract into the first housing 21, such that a side end face of the first housing 21, the second housing 22, and the third housing 23 adjacent to the patient is substantially flush, further reducing the overall volume of the housing assembly 2, which in turn reduces the overall volume of the housing device 100.

In some embodiments, as shown in FIG. 2, when the housing device 100 is fully extended, at least a part of an end of the second housing 22 close to the first housing 21 is located within the first housing 21, and at least a part of an end of the third housing 23 close to the second housing 22 is located within the second housing 22. The above arrangement manner may ensure that a treatment device (e.g., a nozzle) within the housing assembly 2 is not exposed, thereby avoiding entry of foreign matters such as dust, or the like; meanwhile, it may also prevent the first housing 21, the second housing 22, and the third housing 23 from disengaging from each other, and ensure that the second housing 22 can smoothly enter the first housing 21 and the third housing 23 can smoothly enter the second housing 22 when the housing device 100 retracts.

In some embodiments, when the support frame assembly 1 performs a telescopic movement, the second housing 22 and the third housing 23 may perform a telescopic movement along with the support frame assembly 1. That is, the second housing 22 may move along the first direction with the movable base plate 12, and the third housing 23 may move along the first direction with the movable support 13. In this way, the housing device 100 drives the housing assembly 2 to perform the telescopic movement together with the support frame assembly 1, thereby allowing the housing device 100 to perform the linear movement towards and away from a center point of the radiotherapy device, or the like, thereby achieving an increase or a decrease in the volume of the radiotherapy device.

According to some embodiments of the present disclosure, by configuring the three-layer nested housing assembly 2, and by fixing the first housing 21 to the fixed base 11, slidably connecting the second housing 22 to the support frame assembly 1, and fixing the third housing 23 to the movable support 13, the linked extension and retraction of the housing assembly 2 and the support frame assembly 1 is achieved. When the housing device 100 retracts, the second housing 22 and the third housing 23 can successively retract into the first housing 21 and the second housing 22, thereby greatly reducing the overall volume of the housing device 100 and making the foremost end faces substantially flush, effectively releasing space in a treatment area, reducing patient compression and collision risks, meanwhile, ensuring mutual nesting between the housings and preventing disengagement during extension, preventing foreign matters from entering the interior, and improving the safety and reliability of the treatment device.

In some embodiments, as shown in FIGS. 4-6, the drive assembly 3 further includes a third gear 35 and a third rack engaged with the third gear 35, the third rack is disposed in the second housing 22, and the second housing 22 is driven by the third gear 35 and the third rack to move back and forth along the first direction.

The third gear 35 and the third rack are configured to cooperate to drive the second housing 22 to move along the first direction. In some embodiments, the third gear 35 may be disposed on the movable base plate 12, and the third rack is disposed in the second housing 22.

In some embodiments, the third gear 35 and the third rack are engaged with each other, thereby driving the third rack to move through rotation of the third gear, which in turn drives the second housing 22 to move along the first direction.

In some embodiments, as shown in FIGS. 4-6, the third gear 35 may be directly driven by the motor, or may be indirectly driven by the first gear 31 or the second gear 33.

In some embodiments, as shown in FIGS. 4-6, the third gear 35 is driven by the second gear 33, a fourth gear 37, and a fifth gear 38 which are engaged with each other, to allow the rotation direction of the third gear 35 to be consistent with the rotation directions of the first gear 31 and the second gear 33. The fourth gear 37 and the fifth gear 38 serve as intermediate transmission gears, configured to transmit the rotation power of the second gear 33 to the third gear 35, to keep the rotation direction of the third gear 35 consistent with the rotation directions of the first gear 31 and the second gear 33, thereby achieving the linked extension and retraction movement of the movable base plate 12, the movable support 13, and the second housing 22 simultaneously driven by a single motor. In some embodiments, the second gear 33, the fourth gear 37, and the fifth gear 38 are sequentially engaged along the first direction, forming a gear transmission chain, to transmit the rotation power of the second gear 33 to the third gear 35. In some embodiments, the third gear 35 and the fifth gear 38 are coaxially arranged, that is, the third gear 35 rotates along with the fifth gear 38. In some embodiments, the second gear 33, the fourth gear 37, and the fifth gear 38 are gears of the same specification, and the second gear 33, the fourth gear 37, and the fifth gear 38 are linearly distributed along the first direction, thereby avoiding interference of the third rack 36 with the second gear 33, the fourth gear 37, and the fifth gear 38 during movement.

According to some embodiments of the present disclosure, by adding the third gear 35 and the third rack, the second housing 22 can be actively driven to move along the first direction, thereby achieving linked extension and retraction of the second housing 22, the movable base plate 12, and the movable support 13. The structure enhances the controllability and synchronization of the telescopic movement of the housing assembly 2, which helps to optimize stroke matching of each housing, ensuring that when the housing device 100 retracts, the second housing 22 can smoothly retract into the first housing 21, and reliable nesting is achieved during extension, further improving the overall compactness, operational stability, and protective effect of the housing device 100.

In some embodiments, as shown in FIG. 5, the diameter of the third gear 35 is smaller than the diameter of the second gear 33.

Since the distance that the movable support 13 needs to move is greater than the distance that the second housing 22 needs to move, the diameter of the third gear 35 is smaller than the diameter of the second gear 33, to allow the movement speed of the movable support 13 to be greater than the movement speed of the second housing 22. The diameter of the second gear 33 and the diameter of the third gear 35 may be set according to actual needs, to ensure that the movable support 13 and the second housing 22 can simultaneously reach their respective predetermined positions when the housing device 100 retracts.

According to some embodiments of the present disclosure, by configuring the diameter of the third gear 35 to be smaller than the diameter of the second gear 33, under the premise that rotational angular velocities of both are the same, the movement speed of the second housing 22 driven by the third gear 35 is lower than the movement speed of the movable support 13 driven by the second gear 33, thereby matching actual needs where the stroke of the movable support 13 is greater than the stroke of the second housing 22, ensuring that each component simultaneously reaches a predetermined position during extension and retraction, avoiding motion interference or stroke misalignment, and improving the coordination and control accuracy of the extension and retraction movement.

In some embodiments, as shown in FIG. 6, a limiting portion 15 is disposed at a connection end 114 of the fixed base 11 adjacent to the drive frame 200; when the housing device 100 is in the fully retracted state, the movable support 13 is located on a first side 123 of the movable base plate 12, and the movable base plate 12 abuts against the limiting portion 15; when the housing device 100 is in the fully extended state, the movable support 13 is located on a second side 124 of the movable base plate 12; wherein the first side 123 of the movable base plate 12 is adjacent to the drive frame 200 relative to the second side 124 of the movable base plate 12 in the first direction.

The connection end 114 refers to an end of the fixed base 11 connected to the drive frame 200.

The limiting portion 15 is configured to limit the maximum distance of the movable base plate 12 moving towards the drive frame 200. For example, the limiting portion 15 may be an integrally formed boss with the fixed base 11, or a baffle fixed on the fixed base 11 by screws or other fixing manners.

The first side 123 of the movable base plate 12 refers to a side of the movable base plate 12 adjacent to the fixed base 11. The second side 124 of the movable base plate 12 refers to a side of the movable base plate 12 away from the fixed base 11.

The fully retracted state may be a state where the second housing 22 is fully retracted into the first housing 21, and the third housing 23 is fully retracted into the second housing 22.

The fully extended state refers to a state where the housing device 100 may be extended along the first direction to a limit position of the maximum volume.

In some embodiments, as shown in FIGS. 6-8, the limiting portion 15 is disposed at the connection end 114 (i.e., the second end 114) of the fixed base 11 adjacent to the drive frame 200, and the limiting portion 15 is configured to limit the distance of the movable base plate 12 moving towards the drive frame 200.

In some embodiments, when the housing device 100 is in the fully retracted state, the movable support 13 is located on the first side 123 of the movable base plate 12, i.e., the movable support 13 is located on the leftmost side of the movable base plate 12, and the movable base plate 12 abuts against the limiting portion 15. In some embodiments, the drive assembly may drive both the movable support 13 and the movable base plate 12 to move along the direction opposite to the first direction, until the movable base plate 12 abuts against the limiting portion 15.

In some embodiments, when the housing device 100 is in the fully extended state, the movable support 13 is located on the second side 124 of the movable base plate 12, i.e., the movable support 13 is located on the rightmost side of the movable base plate 12. In some embodiments, the drive assembly may drive both the movable support 13 and the movable base plate 12 to move along the direction opposite to the first direction, until the movable support 13 is located on the second side 124 of the movable base plate 12.

According to some embodiments of the present disclosure, by disposing the limiting portion 15 at a first end of the fixed base 11 adjacent to the drive frame 200, the positional relationship is clarified that when the support frame assembly 1 is fully retracted, the movable base plate 12 abuts against the limiting portion 15 and the movable support 13 is located on the first side 123 of the movable base plate 12; and when fully extended, the movable support 13 is located on the second side 124 of the movable base plate 12. This structural arrangement effectively limits the maximum retraction stroke of the movable base plate 12 towards the drive frame 200, thereby preventing component collision or damage caused by excessive retraction, and also ensures clear and reliable positional boundaries of each component during the telescopic movement, and provides a mechanical guarantee for the stable operation and precise positioning of the support frame assembly 1, thereby enhancing the operational safety and control consistency of the housing device 100.

According to some aspects of the present disclosure, a radiotherapy device 20 is also provided. As shown in FIGS. 7-9, the radiotherapy device 20 includes a nozzle and the drive frame 200. The nozzle includes a housing device 100, an ionization chamber, and a particle beam adjustment device. The housing device 100 is driven by the drive frame 200. The housing device 100 includes: the support frame assembly 1, which includes the fixed base 11, the movable base plate 12, and the movable support 13. The movable base plate 12 is disposed on the fixed base 11 and may move back and forth along the first direction. The ionization chamber is installed on the movable base plate 12. The movable support 13 is disposed on the movable base plate 12 and may move back and forth along the first direction. The particle beam adjustment device is installed on the movable support 13. The housing assembly 2, covering at least a part of the support frame assembly 1, is provided with the particle beam outlet 231 and includes three or more housings. At least a part of at least one of the three or more housings may enter or leave at least one other of the three or more housings along the first direction as the movable base plate 12 and/or the movable support 13 moves.

The radiotherapy device is configured to be used for proton radiotherapy. The nozzle is configured to emit a particle beam to treat the patient. The drive frame 200 is configured to drive the housing device 100 to rotate.

In some embodiments, the nozzle is installed on the drive frame 200 of the radiotherapy device and rotates synchronously with the drive frame 200.

The ionization chamber is configured to monitor the proton dose (i.e., the radiation dose of the particle beam) output by the radiotherapy device and spot position information. In some embodiments, the ionization chamber is installed on the movable base plate 12 and may move along the first direction with the movable base plate 12.

The particle beam adjustment device refers to a component configured to adjust and control the shape, intensity, or range of the particle beam to meet treatment needs. In some embodiments, the particle beam adjustment device is installed on the movable support 13 and moves along the first direction with the movable support 13. The particle beam adjustment device includes at least one of a scanning magnet, a range modulator, an AA, or the like.

More descriptions regarding the ionization chamber, the housing device 100, the support frame assembly 1, the housing assembly 2, or the like, may be found elsewhere in the present disclosure (e.g., FIGS. 1-6 and related descriptions thereof).

In some embodiments, during the treatment, the nozzle may rotate along with the rotation of the drive frame 200. In prior art devices, the nozzle is fixed to a C-shaped rail of the guide frame 300. The nozzle and the drive frame 200 move independently, thereby always having a time lag between the movement of the nozzle and the movement of the drive frame 200. Furthermore, the guide frame 300 requires a separate cable system. To simplify the frame design, in some embodiments of the present disclosure, the radiotherapy device directly fixes the nozzle to the drive frame 200, which may ensure the synchronous movement of the nozzle and the drive frame 200, and also greatly optimizes the cabling.

In some embodiments, the nozzle employs a two-layer extension bracket, namely the movable base plate 12 and the movable support 13, which may achieve a larger extended position, thereby allowing the particle beam outlet 231 of the nozzle to be closer to an isocenter (e.g., a patient or the geometric center of a treatment area). For the guide frame 300 that employs a C-shaped frame, the two-layer extension bracket may directly connect the nozzle to the drive frame 200 and remove control motors on the C-shaped frame, thereby fundamentally solving the error and synchronous control problems of the movement of the drive frame 200 and the movement of the nozzle on the C-shaped frame in the related art. The drive frame 200 and the nozzle perform the synchronous rotational movement, thereby reducing waiting time and position deviation.

According to an aspect of the present disclosure, a nozzle is also provided. The nozzle includes the aforementioned housing device 100, the scanning magnet, the ionization chamber, and the RS. The nozzle is installed on the drive frame 200 and rotates along with the drive frame 200. The nozzle is provided with cables, and all the cables are routed through the drive frame 200. That is, the cables on the nozzle are not disposed on the guide frame 300.

According to some embodiments of the present disclosure, by installing the ionization chamber on the movable base plate 12 that is movable along the first direction, and installing the particle beam adjustment device on the movable support 13 that is movable along the first direction, and by configuring the housing assembly 2 with three or more housings capable of mutual nesting and retraction/extension, the linked retraction and extension of internal functional modules and a protective structure of the nozzle is realized. This structure may not only greatly expand the treatment range when the nozzle is extended and significantly reduce the overall volume when retracted, thereby freeing up treatment space and reducing patient discomfort and collision risk, but also allows the housing device 100 to be directly driven by the drive frame 200, avoiding the problem of asynchronous movement between the nozzle and the drive frame 200 in conventional structures, thereby improving treatment accuracy and safety while simplifying the overall machine structure.

In some embodiments, the housing device 100 may rotate relative to at least a part of the drive frame 200.

In some embodiments, a slewing bearing or a rotary slide rail structure may be additionally provided between the housing device 100 and the drive frame 200, thereby allowing the housing device 100 to rotate relative to at least a part of the drive frame 200.

In some implementations, a rotation axis of the housing device 100 is parallel to a rotation axis of the drive frame 200.

In some embodiments, the housing device 100 includes a first locking mechanism (not shown). The first locking mechanism is disposed at a connection end of the housing device 100 and the drive frame 200. The first locking mechanism includes a locking pin, a locking bolt, an electromagnetic lock with a locking flange, or the like, but is not limited thereto. The first locking mechanism includes a locked state and an unlocked state. In the locked state, the housing device 100 and the drive frame 200 are clamped and fixed by the first locking mechanism. When the drive frame 200 drives the housing device 100 to rotate, the housing device 100 does not undergo relative displacement with respect to the intermediate arm 203. In the unlocked state, the housing device 100 may rotate relative to at least a part of the drive frame 200. The locked state and the unlocked state of the first locking mechanism may be manually switched.

According to some embodiments of the present disclosure, by allowing the housing device 100 to independently rotate relative to at least a part of the drive frame 200, a rotational degree of freedom of the housing device 100 itself is increased on the basis of the overall revolution of the drive frame 200, forming a three-dimensional movement group of “revolution+rotation”. The increase in the rotational degree of freedom extends an irradiation coverage range of the radiotherapy beam, thereby improving a target area coverage rate and treatment accuracy for complex shaped tumors. At the same time, a small-range local rotation of the housing may replace a partial large-angle revolution of the drive frame 200, reducing load and wear of the drive frame 200, simplifying an equipment adjustment process, and improving treatment efficiency.

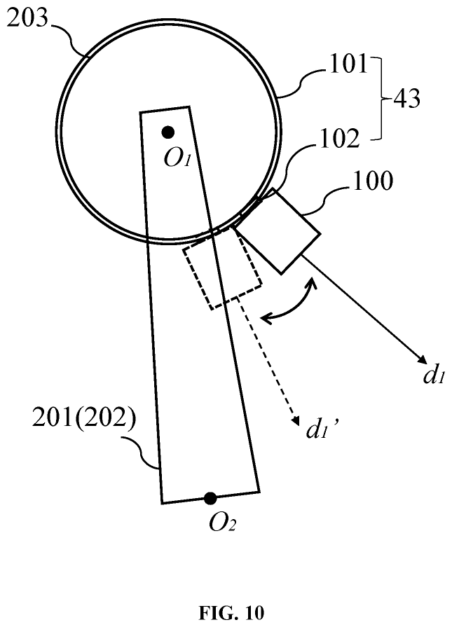

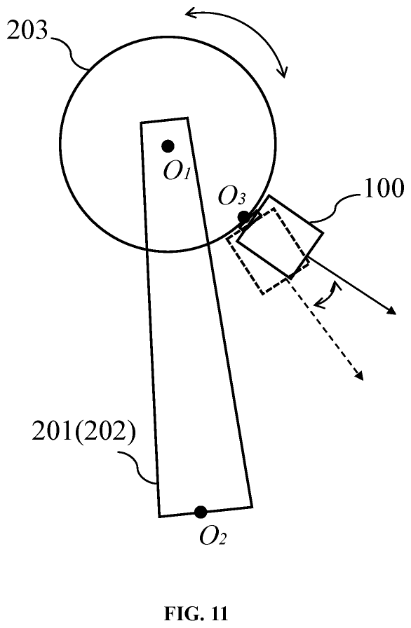

FIG. 10 is a schematic diagram illustrating a housing device rotating around a central axis of an intermediate arm according to some embodiments of the present disclosure. FIG. 11 is a schematic diagram illustrating a housing device rotating around a rotation axis according to some embodiments of the present disclosure.

In some embodiments, as shown in FIGS. 7-11, the drive frame 200 includes a first mechanical arm 201, a second mechanical arm 202, and an intermediate arm 203 in a cylindrical shape. The intermediate arm 203 connects the first mechanical arm 201 and the second mechanical arm 202, and may move along an arc trajectory following the first mechanical arm 201 and the second mechanical arm 202.

The first mechanical arm 201 and the second mechanical arm 202 are rotating arms disposed on two sides of the intermediate arm 203, configured to drive the intermediate arm 203 to rotate. The first mechanical arm 201 and the second mechanical arm 202 are driven by an external power source. The intermediate arm 203 is configured to drive the housing device 100 to rotate.

In some embodiments, the first mechanical arm 201 and the second mechanical arm 202 are located on two sides of the intermediate arm 203 along a length direction of the intermediate arm 203. The first mechanical arm 201 and the second mechanical arm 202 are configured to support the intermediate arm 203 and drive the intermediate arm 203 to rotate.

In some embodiments, the intermediate arm 203 is configured to drive the housing device 100 to move synchronously with the drive frame 200, and a particle accelerator configured to provide a particle beam is installed on the intermediate arm 203.

In some embodiments, the first mechanical arm 201 and the second mechanical arm 202 may rotate about an end away from the intermediate arm 203. As shown in FIGS. 10-11, the first mechanical arm 201 and the second mechanical arm 202 may rotate about the rotation axis 02.

In some embodiments, the housing device 100 may rotate about the intermediate arm 203. The rotation axis of the housing device 100 is parallel to the central axis of the intermediate arm 203. The central axis of the intermediate arm 203 may be a geometric central line of the intermediate arm 203.

In some embodiments, the rotation axis of the housing device 100 is coaxial with the central axis of the intermediate arm 203, and as shown in FIG. 10, the housing device 100 rotates about the central axis 01 of the intermediate arm 203.

The housing device 100 further includes an annular fixing assembly 43. The annular fixing assembly 43 is sleeved on the intermediate arm 203. The annular fixing assembly 43 is configured to connect the housing device 100 and the intermediate arm 203. The annular fixing assembly 43 includes, but is not limited to, a slewing bearing structure, a slide rail and slide block combination, or the like.

In some embodiments, the annular fixing assembly 43 is a slewing bearing structure. An inner ring of the annular fixing assembly 43 is rigidly locked with an outer wall of the intermediate arm 203. In some embodiments, an outer ring of the annular fixing assembly 43 is rigidly locked with the fixed base 11 of the housing device 100. The rigid locking may be achieved by locking with the screw 41 and the pin 42, but is not limited thereto.

In some embodiments, as shown in FIG. 10, the annular fixing assembly 43 includes a third slide rail 401 and a third slide block 402.

In some embodiments, the third slide rail 401 is disposed circumferentially around the intermediate arm 203, the third slide block 402 is fixed on the housing device 100, and the housing device 100 may be slidably disposed on the third slide rail 401 via the third slide block 402, allowing the housing device 100 to slide on the third slide rail 401 to achieve the rotation of the housing device 100 about the central axis of the intermediate arm 203.

The third slide rail 401 refers to an annular rail structure configured to guide the movement of the housing device 100. The third slide block 402 refers to a structural component that cooperates with the third slide rail 401 to achieve the rotation of the housing device 100.

In some embodiments, the housing device 100 may be provided with the third slide block 402, and the housing device 100 may achieve the rotation about the central axis of the intermediate arm 203 via the relative sliding of the third slide block 402 and the third slide rail 401.

According to some embodiments of the present disclosure, by circumferentially disposing the third slide rail 401 on the intermediate arm 203 and slidably disposing the housing device 100 thereon, a rotation guide approach with a simple structure and easy assembly is achieved. The structure allows the housing device 100 to smoothly slide and rotate about the central axis of the intermediate arm 203 along the third slide rail 401, independently adjusting an angle of the housing device 100 on the basis of the revolution of the drive frame 200, thereby achieving precise fine-tuning of a particle beam exit direction with lower mechanical complexity and cost, and further enhancing conformality and flexibility of radiotherapy.

In some embodiments, as shown in FIG. 11, the rotation axis 03 of the housing device 100 is parallel to and not coaxial with the central axis 01 of the intermediate arm 203, and the housing device 100 rotates about the rotation axis 03. The slewing bearing or the bearing structure may be disposed at a connection end of the housing device 100 and the intermediate arm 203, and the slewing bearing or the bearing structure may be connected to the motor rotation shaft of the drive assembly 3, thereby allowing the housing device 100 to rotate about the rotation axis 03 under the drive of the motor. The above arrangement manner may achieve local angle fine-tuning of the housing device 100, and improve treatment accuracy of radiotherapy.

According to some embodiments of the present disclosure, by setting the drive frame 200 as a rotating structure composed of the first mechanical arm 201, the second mechanical arm 202, and the intermediate arm 203 in a cylindrical shape, and by sleeving the housing device 100 on the intermediate arm 203 via the annular fixing assembly 43, direct integration of the housing device 100 and the drive frame 200 is achieved. This arrangement allows the housing device 100 to synchronously move along the arc trajectory with the intermediate arm 203, eliminating the time difference and synchronous control difficulties between the movement of the nozzle and the frame in conventional structures, and at the same time, canceling the C-shaped guide rail and the independent cable system on the guide frame 300, thereby simplifying the overall machine structure, reducing manufacturing and debugging costs, and improving reliability and accuracy of system operation.

In some embodiments, as shown in FIG. 10, the intermediate arm 203 may rotate about a central axis of the intermediate arm 203, thereby driving the housing device 100 on the intermediate arm 203 to rotate about the central axis of the intermediate arm 203. That is, the intermediate arm 203 may rotate relative to the first mechanical arm 201 and the second mechanical arm 202.

In some embodiments, both ends of the intermediate arm 203 along the first direction are rotatably connected to the first mechanical arm 201 and the second mechanical arm 202, respectively. For example, slewing bearings are disposed at both ends of the intermediate arm 203 along the first direction, and the intermediate arm 203 is rotatably connected to the first mechanical arm 201 or the second mechanical arm 202, respectively, via the slewing bearings at the two ends. The above-mentioned rotatable connection may also be implemented in other feasible manners, without being limited thereto.

In some embodiments, the rotation of the intermediate arm 203 about its own central axis may be driven by an independent drive motor.

In some embodiments, the housing device 100 may be fixedly connected to the intermediate arm 203 (e.g., by a screw connection, riveting, welding, or the like), and the rotation of the intermediate arm 203 about the central axis of the intermediate arm 203 may drive the housing device 100 to rotate about the central axis of the intermediate arm 203.

In some embodiments, when the intermediate arm 203 is not required to rotate about its own axis, a locking pin, a locking flange, or an electromagnetic brake may be disposed at the connection mating positions of the slewing bearings at both ends of the intermediate arm 203 with the first mechanical arm 201 and the second mechanical arm 202, thereby circumferentially locking the intermediate arm 203 with the first mechanical arm 201 and the second mechanical arm 202, and limiting the relative rotation between the intermediate arm 203, the first mechanical arm 201, and the second mechanical arm 202.

In some embodiments, the intermediate arm 203 includes a second locking mechanism (not shown in figures). The second locking mechanism is disposed at the connection positions of both ends of the intermediate arm 203 along the first direction with the first mechanical arm 201 and the second mechanical arm 202. The structure and principle of the second locking mechanism are similar to those of the first locking mechanism. More descriptions regarding the specific details may be found in the preceding related descriptions.

According to some embodiments of the present disclosure, the rotation of the intermediate arm 203 about its own central axis drives the housing device 100 to synchronously rotate, thereby eliminating the need to additionally dispose an extra rotary guide mechanism between the housing device 100 and the intermediate arm 203, simplifying the mechanical design, and simultaneously maintaining a high degree of synchronization between the rotation of the housing device 100 and the rotation of the intermediate arm 203 about its own central axis, which further expands the regulation range of the particle beam emission angle based on the overall orbital rotation of the drive frame 200, and improves the flexibility and conformality of the radiotherapy.

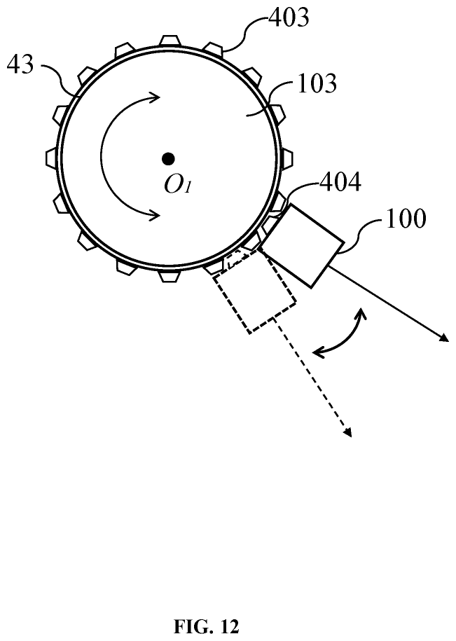

FIG. 12 is a schematic diagram illustrating positions of a gear set and an annular rack according to some embodiments of the present disclosure.

In some embodiments, as shown in FIGS. 1-6 and FIG. 12, the housing device 100 further includes the drive assembly 3. The drive assembly 3 includes the first gear 31, the second gear 33, a gear set 404, the first rack 32 disposed on the fixed base 11, the second rack 34 disposed on the movable support 13, and an annular rack 403 disposed on the annular fixing assembly 43. The first rack 32 and the second rack 34 are engaged with the first gear 31 and the second gear 33 respectively, the extension direction of the first rack 32 and the extension direction of the second rack 34 are both parallel to the first direction, the movable base plate 12 is driven by the first gear 31 and the first rack 32 to move back and forth along the first direction, and the movable support 13 is driven by the second gear 33 and the second rack 34 to move back and forth along the first direction. The annular rack 403 is engaged with a gear of the gear set 404, the annular rack 403 is wound around an outer peripheral side of the annular fixing assembly 43, and the housing device 100 is driven by the annular rack 403 and the gear set 404 to rotate around the intermediate arm 203.

More descriptions regarding the drive assembly 3, the first gear 31, the first rack 32, the second gear 33, the second rack 34, the fixed base 11, the movable base plate 12, and the movable support 13 may be found elsewhere in the present disclosure (e.g., FIGS. 1-6 and related descriptions thereof).

The gear set 404 is configured to output the rotational movement of the motor in the drive assembly 3. The gear set 404 is constituted by a plurality of mutually engaged gears. For example, the gear set 404 may be a planetary gearbox.

In some embodiments, the gear set 404 may be connected to the rotating shaft of the motor. In some embodiments, the gear set 404 may be integrated on an output shaft of the first gear 31 or the second gear 33.

The annular rack 403 is configured to transmit the rotational movement of the gear set 404 to the intermediate arm 203, thereby causing the housing assembly to perform a relative rotational movement with respect to the intermediate arm 203.

In some embodiments, the annular rack 403 may be wound around the outer peripheral side of the annular fixing assembly 43, and fixedly connected to the outer peripheral side of the annular fixing assembly 43. Fixed connection may be implemented by manners such as welding, bolted fixing, or the like; however, it is not limited thereto.

According to some embodiments of the present disclosure, by additionally disposing the annular rack 403 and the gear set 404, the telescopic drive and the rotary drive are integrated into the same drive assembly 3, without the need to additionally dispose an independent rotary power source, thereby improving the structural compactness of the housing device 100. The above-mentioned arrangement realizes the rotation of the housing device 100 relative to the intermediate arm 203, and by means of a single drive assembly 3, a dual-layer telescopic movement and rotation are achieved, thereby increasing the degrees of freedom of movement of the housing device 100, and further improving the treatment flexibility of the radiotherapy device.

In some embodiments, the drive assembly 3 further includes a motor (not shown in figures), a first electrically controlled clutch (not shown in figures), and a second electrically controlled clutch (not shown in figures). The first gear 31 and the second gear 33 are coaxially connected to the rotating shaft of the motor via the first electrically controlled clutch; and the gear set 404 is coaxially connected to the rotating shaft of the motor via the second electrically controlled clutch.

More descriptions regarding the motor may be found elsewhere in the present disclosure (e.g., FIGS. 4-6 and related descriptions thereof).

The first electrically controlled clutch refers to a clutch device configured to control a linear telescopic movement. For example, the first electrically controlled clutch may be an electromagnetic clutch, or the like.

In some embodiments, a central axis of the first gear 31, a central axis of the second gear 33, a central axis of the first electrically controlled clutch, and a central axis of the rotating shaft of the motor are collinear. When the first electrically controlled clutch is connected, the first electrically controlled clutch transmits the torque of the motor to the first gear 31 and the second gear 33 and drives the first gear 31 and the second gear 33 to rotate, thereby driving the first rack 32 and the second rack 34 to perform the linear telescopic movement along the first direction. When the first electrically controlled clutch is disconnected, the torque of the motor cannot be transmitted to the first gear 31 and the second gear 33, and the first gear 31 and the second gear 33 are in an idle or locked state.

The second electrically controlled clutch refers to a clutch device configured to control the rotational movement. For example, the second electrically controlled clutch may be an electromagnetic clutch, or the like.

In some embodiments, a central axis of the gear set 404, a central axis of the second electrically controlled clutch, and a central axis of the rotating shaft of the motor are collinear. When the second electrically controlled clutch is connected, the motor drives the gear set 404 to rotate, thereby driving the annular rack 403 to perform the rotational movement. When the first electrically controlled clutch is disconnected, the gear set 404 is in an idle or locked state.

According to some embodiments of the present disclosure, by disposing the first electrically controlled clutch and the second electrically controlled clutch, the same motor may switch power output between a telescopic mode and a rotary mode, respectively: when the first electrically controlled clutch is engaged, the motor drives the first gear 31 and the second gear 33 to realize the telescopic movement of the housing device 100; and when the second electrically controlled clutch is engaged, the motor drives the gear set 404 and the annular rack 403 to realize the rotational movement of the housing device 100. This structure realizes “multi-purpose operation”, and enables the driving of two degrees of freedom, namely telescopic movement and rotation, with only a single motor system, thereby greatly reducing the overall weight and complexity of the nozzle, simplifying the design of a control system, avoiding synchronization control problems associated with multi-motor coordination, and simultaneously reducing equipment manufacturing costs and energy consumption.

In some embodiments, an output power of the motor is nonlinearly positively correlated with an extension length of the housing device 100 along the first direction.

The output power of the motor refers to the output power of the motor of the drive assembly 3 when operating.

The extension length refers to a variable distance of the housing device 100 along the first direction between the fully retracted state and the fully extended state.

In some embodiments, the extension length may be obtained in a plurality of manners. For example, the extension length may be directly obtained by a visual sensor (e.g., a laser ranging sensor, or the like) installed in the housing device 100. As another example, the extension length may be determined by a transmission model, including: obtaining a count of rotations and an angle of the motor by a motor encoder; and determining the extension length by the transmission model.

The motor encoder refers to a sensor installed on the rotating shaft of the motor, configured to convert the rotational displacement of the motor into an electrical signal to measure the count of rotations and the angle of the rotating shaft of the motor.

The transmission model refers to a mathematical model configured to determine the movement distance of the racks. The transmission model may be preset based on experience. The movement distance of the racks is the extension length of the housing device 100. The transmission model may be preset based on experience.

In some embodiments, the extension length may be obtained in other feasible manners; the present disclosure is not limited thereto.

In some embodiments, the output power of the motor may be determined by a plurality of manners. For example, a manner for determining the output power of the motor includes: determining the output power of the motor based on the extension length by a mapping function or a preset table. The mapping function may be a quadratic function, an exponential function, or the like. The preset table includes a mapping relationship between the extension length and the output power. The mapping function and the preset table may be obtained by historical data or experimental data.

In some embodiments, the output power of the motor may be obtained by the preset table. The preset table includes the mapping relationship between the extension length and the output power of the motor. The mapping relationship may be obtained by experimental data or historical data. For example, for a certain extension length, by continuously adjusting the output power of the motor through an experiment, the output power when no abnormality occurs in the housing device 100 is taken as the output power corresponding to the extension length. The abnormality includes stuttering, severe shaking, or the like.

In some embodiments, the manner for determining the output power of the motor includes: determining a rotational inertia of the housing device 100 based on the extension length; taking a product of the rotational inertia of the housing device 100 and an angular acceleration of the motor as a target torque; and determining an output current of the motor based on the target torque. In a case where the voltage remains unchanged, the output power of the motor is positively correlated with the output current of the motor. The angular acceleration of the motor may be measured by an angular acceleration sensor, or the like.

In some embodiments, according to a basic formula of rotational inertia, it may be determined that the rotational inertia of the housing device 100 is nonlinearly positively correlated with the extension length (approximately quadratically positively correlated). The basic formula of rotational inertia may be expressed as Formula (1):

J=mr², (1)

where J represents the rotational inertia, m represents the mass of a point mass, and r represents the distance from the point mass to the rotating shaft. The m of the housing device 100 remains unchanged, and r is positively correlated with the extension length. Therefore, the rotational inertia of the housing device 100 is approximately quadratically positively correlated with the extension length.

It should be noted that the housing device 100 is configured as a multi-body dynamic system with a variable mass distribution. While the formula (1) provides a fundamental basis, the effective rotational inertia J_{eff} of the housing device 100 is more accurately modeled as a function of the extension length L. The effective rotational inertia J_{eff} may be determined by Formula (2). Formula (2) is shown below:

$${J}_{eff} ( L ) = {J}_{b a s e} + {\int }_{0}^{L} {x}^{2} \cdot ρ ( x , L ) dx + Δ {J}_{c a b l e} ( L ) , \qquad ( 2 )$$

where J_{base} represents the static inertia of the rotation frame, J_{base} may be determined through formula (1); ρ(x, L) denotes the non-homogeneous mass density distribution of the extending segments, and ΔJ_{cable}(L) accounts for the dynamic inertia contribution of the internal cabling and drag compensation mechanisms. This approach ensures that the target torque calculation compensates for the non-linear inertia shift caused by the migration of the center of gravity and the inertial effects of internal auxiliary components during the stroke.

The target torque refers to a predicted adjusted torque of the motor of the drive assembly 3. In some embodiments, the output current of the motor is approximately linearly correlated with the target torque. The adjustment ratio of the output current of the motor may be consistent with the adjustment ratio of the target torque. By adjusting the output current of the motor, an adjustment of the output power of the motor is achieved.

In some embodiments, the output current of the motor may be determined by Formula (3). Formula (3) is shown below:

T≈KΦI, (3)

where T denotes the target torque; K denotes the structural constant of the motor; Φ denotes the main magnetic flux of the motor; and I denotes the output current of the motor. The structural constant of the motor and the main magnetic flux of the motor may be obtained by a user terminal.

During the retraction and extension process of the housing device 100, the center of gravity of the housing device 100 changes. If a fixed torque output is adopted, the housing device 100 is prone to “insufficient torque” during an extension process, causing slow rotation, step loss, or the like; or prone to “excess torque” in a retracted state, causing impact and energy consumption waste. According to some embodiments of the present disclosure, by adjusting motor power, the torque of the motor may match a nonlinear increase in the rotational inertia of the housing device 100 resulting from the center of gravity position change during the retraction and extension process, thereby achieving on-demand energy supply, and further ensuring smooth and precise rotational movement, thereby improving motor energy efficiency and reducing mechanical wear.

In some embodiments, as shown in FIGS. 7 to 9, the radiotherapy device further includes the guide frame 300, wherein the housing device 100 is movably disposed on the guide frame 300. The guide frame 300 is an arcuate frame, wherein an included angle between the first direction and a second direction is in a range of 0 to 15°. The drive frame 200 includes the first mechanical arm 201, the second mechanical arm 202, and the intermediate arm 203. The intermediate arm 203 connects the first mechanical arm 201 and the second mechanical arm 202 and moves along the arc trajectory following the first mechanical arm 201 and the second mechanical arm 202. The housing device 100 is fixedly connected to the intermediate arm 203, and moves along the guide frame 300 driven by the intermediate arm 203.

More descriptions regarding the first mechanical arm 201, the second mechanical arm 202, the intermediate arm 203, and the arc trajectory movement may be found elsewhere in the present disclosure (e.g., FIGS. 1 to 9 and related descriptions thereof).

The guide frame 300 is configured to guide an irradiation direction of the particle beam during a radiotherapy process.

The radial direction of the arcuate frame may be a direction perpendicular to an arcuate surface of the arcuate frame.

The second direction is a radial direction of the arcuate frame. The radial direction of the arcuate frame may be a direction passing through the center of the arcuate frame. For example, as shown in FIG. 7, the second direction may be a direction parallel to the axis D-D, and the angle between the first direction and the second direction is a, where a ranges from 0° to 15°. By such arrangement, the first direction may substantially coincide with the second direction, thereby facilitating the movement of the housing device on the arcuate frame.

In some embodiments, an inner side of the guide frame 300 (i.e., a side facing the patient) may be provided with an arcuate fourth slide rail, and a corresponding fourth slide block may be installed on the housing device 100. The intermediate arm 203 moves along the arc trajectory driven by the drive frame 200. Since the housing device 100 is fixedly connected to the intermediate arm 203, the housing device 100 moves synchronously with the intermediate arm 203. Furthermore, the housing device 100, in cooperation with the fourth slide rail and the fourth slide block, slides along an arcuate trajectory of the guide frame 300.

According to some embodiments of the present disclosure, by setting the included angle between the extension direction (i.e., the first direction) of the housing device 100 and the radial direction (i.e., the second direction) of the arcuate guide frame 300 to be in a range of 0 to 15°, the housing device 100, when following the drive frame 200 to move along the arc trajectory and extending/retracting along the first direction, may better adapt to a spatial layout around a top of a treatment bed, thereby avoiding interference with a patient or an operator, and simultaneously optimizing a positioning path of the particle beam outlet 231 relative to an isocenter, which improves movement smoothness and positioning accuracy of the nozzle on the arcuate guide frame 300, further increases an effective space of a treatment area, and reduces a collision risk.

In some embodiments, the radiotherapy device further includes a particle accelerator, wherein the particle accelerator is installed on the intermediate arm 203; and cables of the particle beam adjustment device, the ionization chamber, and the particle accelerator are all routed through the drive frame 200.

The particle accelerator refers to a device configured to generate a particle beam for provision to the nozzle.

More descriptions regarding the particle beam adjustment device and the ionization chamber may be found elsewhere in the present disclosure (e.g., FIGS. 7 to 9 and related descriptions thereof).

In some embodiments, the drive frame 200 is a hollow structure or is provided with a channel for the passage of cables therein.

According to some embodiments of the present disclosure, by directly installing the particle accelerator on the intermediate arm 203 of the drive frame 200 and by routing the cables of the particle beam adjustment device, the ionization chamber, and the particle accelerator uniformly through the drive frame 200, electrical connections between the nozzle and the drive frame 200 may be highly integrated, thereby effectively simplifying an overall wiring structure and reducing manufacturing and debugging costs. Simultaneously, reliability concerns caused by exposed cables or drag chain movement are eliminated, thereby further improving overall operational stability and safety of the radiotherapy device.

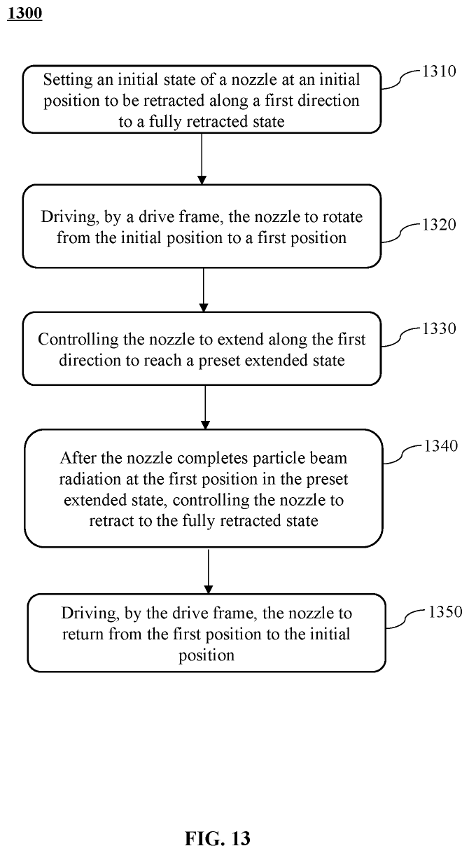

FIG. 13 is a flowchart illustrating an exemplary method for movement control of a nozzle according to some embodiments of the present disclosure. As shown in FIG. 13, a process 1300 is an exemplary process of the method for movement control of the nozzle, and the process 1300 includes operations 1310-1350. The operations 1310-1350 may be executed by the processor. The processor may include one or more of a microcontroller (MCU), an embedded processor, a graphics processing unit (GPU), or the like.

In 1310, an initial state of the nozzle is set at an initial position to be retracted along a first direction to a fully retracted state.

The initial position refers to a reference position of the nozzle before moving. The initial state refers to the state of the nozzle before moving.

In some embodiments, the processor may issue a retraction instruction to the drive assembly 3, thereby causing the movable base plate 12 and the movable support 13 to move along the first direction towards a side away from a patient, until the movable base plate 12 abuts against the limiting portion 15, and the movable support 13 is located at the first side 123 of the movable base plate 12, at which point the housing device 100 is in the fully retracted state, thereby completing the setup of the initial state.

In 1320, the nozzle is driven to rotate from the initial position to a first position by the drive frame.

The first position refers to a position to be reached. In some embodiments, the first position may be set according to treatment needs (e.g., a patient height, a treatment area), or the like.

In some embodiments, the processor, according to the set first position, sends a rotation instruction to the motor (e.g., a motor of the drive assembly 3 or an external power source), thereby driving related rotating components (e.g., at least one of the housing device 100, the intermediate arm 203, the first mechanical arm 201, and the second mechanical arm 202) to rotate until the nozzle reaches the first position.

In 1330, the nozzle is controlled to extend along the first direction to reach a preset extended state.

The preset extended state refers to a state where the housing device 100 of the nozzle reaches a target extension length. In some embodiments, the target extension length may be determined according to a treatment plan. The processor may obtain the treatment plan uploaded by a user via the user terminal.

In some embodiments, the processor sends an extension instruction to the drive assembly 3, thereby controlling the motor to rotate and driving the movable base plate 12 and the movable support 13 to move along the first direction to the target extension length.

In 1340, after the nozzle completes particle beam radiation at the first position in the preset extended state, the nozzle is controlled to retract to the fully retracted state.

The particle beam radiation refers to a process in which a particle beam, adjusted by a particle beam adjustment device, is emitted through the particle beam outlet 231 of the housing device 100 and guided to a patient treatment area.

In some embodiments, the processor sends a retraction instruction to the drive assembly 3, thereby controlling the motor to rotate in reverse and driving the movable base plate 12 and the movable support 13 to move along the first direction toward the side away from the patient until the movable base plate 12 abuts against the limiting portion 15 and the movable support 13 is located on the first side 123 of the movable base plate 12.

In 1350, the nozzle is driven by the drive frame to return from the first position to the initial position.

In some embodiments, the processor sends an instruction to the drive motor of the drive frame 200, thereby causing the drive frame 200 to rotate in reverse and driving the nozzle to return from the first position to the initial position.

More descriptions may be found elsewhere in the present disclosure (e.g., FIGS. 1-12 and related descriptions thereof).

According to an aspect of the present disclosure, a method for movement of the nozzle is also provided. The method includes: setting the initial state of the nozzle at the initial position to be retracted along a front-back direction to a final state; according to a predetermined position to be reached by the nozzle, rotating the drive frame 200 and driving the nozzle to rotate from the initial position to the first position, and then extending forward from the first position to reach the predetermined position; and after completing particle beam radiation at the predetermined position, retracting the nozzle to the first position.

In some embodiments, the method for movement of the nozzle further includes: after retracting the nozzle to the first position, rotating the drive frame 200, thereby causing the nozzle to return to the initial position.

According to some embodiments of the present disclosure, by first placing the nozzle in the fully retracted state and then performing a rotational movement, collision between the nozzle and the patient or peripheral equipment is effectively avoided, thereby improving the safety of the treatment process; after reaching the first position, controlling the nozzle to extend to the preset extended state allows the particle beam outlet to precisely approach the treatment area, thereby both ensuring effective coverage of the treatment range and minimizing space occupation of the nozzle within the treatment area, and reducing the patient's sense of compression; after completing the treatment, first retracting and then rotating back to the initial position further optimizes the movement path, simplifies the operation process, and improves treatment efficiency and the safety of device operation.

Although the embodiments of the present disclosure have been shown and described above, it is to be understood that the above-described embodiments are illustrative and are not to be construed as limiting the present disclosure. A person of ordinary skill in the art may make changes, modifications, substitutions, and variations to the above-described embodiments without departing from the principle and spirit of the present disclosure and within the scope of the present disclosure, and all such changes are intended to be within the scope of the claims of the present disclosure.

Meanwhile, the present disclosure uses specific words to describe the embodiments of the present disclosure. For example, “an embodiment”, “one embodiment”, and/or “some embodiments” refer to a certain feature, structure, or characteristic related to at least one embodiment of the present disclosure. Therefore, it should be emphasized and noted that “one embodiment” or “an embodiment” or “an alternative embodiment” mentioned twice or more times at different places in the present disclosure does not necessarily refer to the same embodiment. Furthermore, certain features, structures, or characteristics in one or more embodiments of the present disclosure may be suitably combined.

Moreover, unless expressly specified in the claims, the order of processing elements and sequences, the use of numerical letters, or the use of other names described in the present disclosure is not intended to limit the order of the processes and manners of the present disclosure. Although some presently useful embodiments of the present disclosure are discussed by various examples in the foregoing disclosure, it should be understood that such details are for illustrative purposes only, and the appended claims are not limited to the disclosed embodiments. Rather, the claims are intended to cover all modifications and equivalent combinations that fall within the spirit and scope of the embodiments of the present disclosure. For example, although the system components described above may be implemented by hardware devices, they may also be implemented by software solutions only, such as installing the described system on an existing server or mobile device.

Similarly, it should be noted that, in order to simplify the description of the present disclosure and thereby facilitate an understanding of one or more embodiments of the present disclosure, various features may sometimes be grouped into a single embodiment, figure, or description thereof in the foregoing description of the embodiments of the present disclosure. However, this manner of disclosure does not imply that the subject matter of the present disclosure requires more features than those recited in the claims. In fact, an embodiment may have fewer features than all the features of a single embodiment of the foregoing disclosure.

In some embodiments, numbers describing the quantity of components or attributes are used. It should be understood that such numbers for describing the embodiments may be used in some examples with modifiers such as “about”, “approximate”, or “substantially”. Unless otherwise specified, “about”, “approximate”, or “substantially” indicate that the recited number allows for a variation of ±20%. Accordingly, in some embodiments, numerical parameters used in the present disclosure and the claims are approximate values, and the approximate values may vary according to characteristics required by individual embodiments. In some embodiments, the numerical parameters should consider specified significant digits and be applied with a generally accepted manner of rounding. Although numerical ranges and parameters for defining the scope of the present disclosure in some embodiments of the present disclosure are the approximate values, in specific embodiments, the settings of such values may be made as precise as practicable within a practical range.

Each patent, patent application, patent application publication, and other material cited in the present disclosure, such as articles, books, disclosures, publications, documents, or the like, is hereby incorporated by reference in its entirety into the present disclosure. This excludes application history documents that are inconsistent with or conflict with the content of the present disclosure, and also excludes documents that limit the broadest scope of the claims of the present disclosure (currently or subsequently attached to the present disclosure). It should be noted that if there is any inconsistency or conflict between the descriptions, definitions, and/or usages of terms in subsidiary materials of the present disclosure and the content described in the present disclosure, the descriptions, definitions, and/or usages of terms of the present disclosure shall prevail.

What is claimed is:

1. A housing device, movably arranged on a guide frame of a radiotherapy device and driven by a drive frame of the radiotherapy device, wherein the housing device comprises:
a support frame assembly, including a fixed base, a movable base plate, and a movable support, wherein the movable base plate is disposed on the fixed base and capable of moving back and forth along a first direction, and the movable support is disposed on the movable base plate and capable of moving back and forth along the first direction; and
a housing assembly, covering at least a part of the support frame assembly, wherein the housing assembly is provided with a particle beam outlet and includes three or more housings, wherein at least a part of at least one of the three or more housings is capable of entering or leaving at least one other of the three or more housings along the first direction as the movable base plate and/or the movable support moves.

2. The housing device according to claim 1, further comprising a drive assembly, wherein the movable base plate and the movable support are driven by the drive assembly to move back and forth along the first direction.

3. The housing device according to claim 2, wherein the drive assembly includes a first gear and a second gear, a first rack disposed on the fixed base, and a second rack disposed on the movable support, the first rack and the second rack are engaged with the first gear and the second gear respectively, an extension direction of the first rack and an extension direction of the second rack are both parallel to the first direction, the movable base plate is driven by the first gear and the first rack to move back and forth along the first direction, and the movable support is driven by the second gear and the second rack to move back and forth along the first direction.

4. The housing device according to claim 3, wherein a diameter of the second gear is larger than a diameter of the first gear.

5. The housing device according to claim 3, wherein the housing assembly includes a first housing, a second housing, and a third housing sequentially arranged along the first direction, the first housing is fixedly connected to the fixed base, the second housing is slidably connected to the support frame assembly, and the third housing is fixedly connected to the movable support; and
when the housing device is retracted, the second housing retracts into the first housing, and the third housing retracts into the second housing.

6. The housing device according to claim 5, wherein the drive assembly further includes a third gear and a third rack engaged with the third gear, the third rack is disposed in the second housing, and the second housing is driven by the third gear and the third rack to move back and forth along the first direction.

7. The housing device according to claim 6, wherein a diameter of the third gear is smaller than a diameter of the second gear.

8. The housing device according to claim 5, wherein a pulley is disposed on the movable support, a guide rail slidably connected with the pulley is disposed on the second housing, and the second housing performs a linear movement along the first direction driven by the pulley and the guide rail.

9. The housing device according to claim 5, wherein a limiting portion is disposed at a connection end of the fixed base adjacent to the drive frame;
when the housing device is in a fully retracted state, the movable support is located on a first side of the movable base plate, and the movable base plate abuts against the limiting portion; and
when the housing device is in a fully extended state, the movable support is located on a second side of the movable base plate;
wherein the first side of the movable base plate is adjacent to the drive frame relative to the second side of the movable base plate in the first direction.

10. The housing device according to claim 1, further comprising a fixing assembly, wherein the support frame assembly is fixedly connected to the drive frame via the fixing assembly, the support frame assembly further includes a support frame, the housing assembly includes a first housing, a second housing, and a third housing sequentially arranged along the first direction, and the support frame is connected to the fixed base and the first housing.

11. The housing device according to claim 1, wherein the support frame assembly further includes a first slide rail and a first slide block slidably connected, and a second slide rail and a second slide block slidably connected, the movable base plate and the fixed base are slidably connected via the first slide rail and the first slide block, and the movable base plate and the movable support are slidably connected via the second slide rail and the second slide block.

12. A radiotherapy device, comprising a nozzle and a drive frame, wherein the nozzle includes a housing device, an ionization chamber, and a particle beam adjustment device; the housing device is driven by the drive frame, the housing device including:
a support frame assembly, including a fixed base, a movable base plate and a movable support, wherein the movable base plate is disposed on the fixed base and capable of moving back and forth along a first direction, the ionization chamber is installed on the movable base plate, the movable support is disposed on the movable base plate and capable of moving back and forth along the first direction, and the particle beam adjustment device is installed on the movable support; and
a housing assembly, covering at least a part of the support frame assembly, the housing assembly being provided with a particle beam outlet and including three or more housings, wherein at least a part of at least one of the three or more housings is capable of entering or leaving at least one other of the three or more housings along the first direction as the movable base plate and/or the movable support moves.

13. The radiotherapy device according to claim 12, wherein the housing device is capable of rotating relative to at least a part of the drive frame.

14. The radiotherapy device according to claim 13, wherein the drive frame includes a first mechanical arm, a second mechanical arm, and an intermediate arm in a cylindrical shape, the intermediate arm connects the first mechanical arm and the second mechanical arm, and the intermediate arm is capable of moving along an arc trajectory following the first mechanical arm and the second mechanical arm; and
the housing device further includes an annular fixing assembly, and the fixing assembly is sleeved on the intermediate arm.

15. The radiotherapy device according to claim 14, wherein the housing device further includes a drive assembly; the drive assembly includes a first gear, a second gear, a gear set, a first rack disposed on the fixed base, a second rack disposed on the movable support, and an annular rack disposed on the annular fixing assembly;
the first rack and the second rack are engaged with the first gear and the second gear respectively, an extension direction of the first rack and an extension direction of the second rack are both parallel to the first direction, the movable base plate is driven by the first gear and the first rack to move back and forth along the first direction, and the movable support is driven by the second gear and the second rack to move back and forth along the first direction; and
the annular rack is engaged with a gear of the gear set, the annular rack is wound around an outer peripheral side of the annular fixing assembly, and the housing device is driven by the annular rack and the gear set to rotate around the intermediate arm.

16. The radiotherapy device according to claim 15, wherein the drive assembly further includes a motor, a first electrically controlled clutch and a second electrically controlled clutch;
the first gear and the second gear are coaxially connected to a rotating shaft of the motor via the first electrically controlled clutch; and
the gear set is coaxially connected to the rotating shaft of the motor via the second electrically controlled clutch.

17. The radiotherapy device according to claim 16, wherein an output power of the motor is nonlinearly positively correlated with an extension length of the housing device along the first direction.

18. The radiotherapy device according to claim 12, further comprising a guide frame, wherein the housing device is movably disposed on the guide frame;
the guide frame is an arcuate frame, an included angle between the first direction and a second direction is in a range of 0-15°, and the second direction is a radial direction of the arcuate frame;
the drive frame includes a first mechanical arm, a second mechanical arm, and an intermediate arm, the intermediate arm connects the first mechanical arm and the second mechanical arm and moves along an arc trajectory following the first mechanical arm and the second mechanical arm; and
the housing device is fixedly connected to the intermediate arm, and the housing device moves along the guide frame driven by the intermediate arm.

19. The radiotherapy device according to claim 18, further comprising: a particle accelerator, wherein the particle accelerator is installed on the intermediate arm; cables of the particle beam adjustment device, the ionization chamber, and the particle accelerator are all routed through the drive frame.

20. A method for movement control of a nozzle, wherein the method comprises:
setting an initial state of the nozzle at an initial position to be retracted along a first direction to a fully retracted state;
driving, by a drive frame, the nozzle to rotate from the initial position to a first position;
controlling the nozzle to extend along the first direction to reach a preset extended state;
after the nozzle completes particle beam radiation at the first position in the preset extended state, controlling the nozzle to retract to the fully retracted state; and
driving, by the drive frame, the nozzle to return from the first position to the initial position.
